SEQUENCE LISTING (1 sequence)

SEQ ID NO: 1 - protein, 401 aa, synthetic construct
MGVKVLFALICIAVAEAKPTENNEDFNIVAVASNFATTDLDADRGKLPGEKLPLEVLKELEANARKAGCTRGCLICLSHIKCTPKMKKFIPGRCHTYEGDKESAQGGIGEAIDDIPEIPGFKDLEPIEQFIAQVDLCVDCTTGCLKGLANVQCSDLLKKWLPQRCATFASKIQGQVDKIKGAGDDGSVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

(19) United States
(12) Patent Application Publication
(10) Pub. No.: US 2025/0327753 A1
(43) Pub. Date: Oct. 23, 2025

(54) NONINVASIVE MONITORING OF GENE EXPRESSION IN THE BRAIN WITH SYNTHETIC SERUM MARKERS

(71) Applicant: William Marsh Rice University, Houston, TX (US)

(72) Inventors: Jerzy Olgierd Szablowski, Houston, TX (US); Sangsin Lee, Houston, TX (US)

(21) Appl. No.: 18/869,209

(22) PCT Filed: May 30, 2023
(86) PCT No.: PCT/US2023/067627

§ 371 Date: Nov. 25, 2024

(51) Int. Cl. G01N 21/76; C12N 9/02; C12Q 1/26; G01N 33/49

(52) U.S. Cl. CPC G01N 21/763; C12N 9/0069; C12Q 1/26; G01N 33/49; C07K 2319/02; C07K 2319/30; C07K 2319/61; C12Y 113/12005; G01N 2333/90241

(57) ABSTRACT

A platform is described that enables multiplexed, noninvasive, and site-specific monitoring of brain gene expression through a class of engineered reporters, referred to herein as Released Markers of Activity (RMAs). Instead of detecting gene expression in the less accessible brain, RMA reporters exit the brain into the blood, where they can be measured with biochemical techniques. When placed under a promoter upregulated by neuronal activity, RMAs may be used to measure neuronal activity in specific brain regions with a simple blood draw. As discussed herein, the present approaches provide a noninvasive paradigm for repeatable and multiplexed monitoring of gene expression in an intact brain with sensitivity that is currently unavailable through other noninvasive gene expression reporter systems.

### CROSS-REFERENCE TO RELATED APPLICATIONS

This application claims priority to PCT application No. PCT/US23/67627, entitled “Noninvasive Monitoring Of Gene Expression In The Brain With Synthetic Serummarkers,” filed May 30, 2023, which claims benefit of U.S. Provisional Application No. 63/346,958, entitled “Noninvasive Monitoring of Gene Expression in the Brain”, filed May 30, 2022, which are incorporated by reference in all their entirety herein.

### STATEMENT OF GOVERNMENT SUPPORT

This invention was made with government support under Grant Number R21EB033059 awarded by the National Institutes of Health. The government has certain rights in the invention.

### TECHNICAL FIELD

The subject matter disclosed herein relates to non-invasive techniques for mapping gene expression.

### BACKGROUND

Monitoring gene expression dynamics in the living brain is useful for studying the brain's cellular activity, understanding complex cognitive behaviors, and controlling the onset of neurological diseases. However, the delicate and intricate architecture of the brain poses challenges to tracking gene expression in vivo and requires noninvasive analytical methods that are both sensitive and specific.

Various noninvasive technologies have exploited the use of genetically encodable reporters for mapping gene expression in the intact brain. Although they vary in their operational complexity, each methodology must contend with trade-offs in terms of noninvasiveness, sensitivity, specificity, and depth. For example, magnetic resonance imaging (MRI) can visualize gene expression throughout the whole brain with sub-millimeter spatial resolution using genetically encoded contrast agents, including iron-binding and non-metallic reporters. However, reporter sensitivity and contrast resolution are marred by competing background signals from surrounding tissue. Additionally, efforts to measure endogenous promoter activity, including that of the immediate early gene (IEG) Fos, have yet to yield success. In ultrasound imaging, great strides have been made in enhancing contrast through the application of genetically encoded air-filled gas vesicles (GVs), which have also been shown to augment signal contrast in MRI. Still, the use of GVs has several drawbacks, including having to deliver ultrasound to each GV variant, which is difficult in thick skulls, large transgene sizes, and limited multiplexing capabilities. Lastly, optical imaging systems, such as optoacoustic and fluorescence imaging utilize reporters to enable brain imaging with subcellular spatial resolution at millisecond timescales and have been widely adopted for in vivo interrogations of neuronal activity. However, the heterogeneity of the brain as well as the opacity of the skull inherently limit the depth at which optical modalities can probe gene expression activities. Because of the strong light scattering and absorption properties of brain tissue, optical systems face the formidable challenge of imaging subcortical and deep cortical regions, particularly in large brains. While recent improvements in bioluminescence imaging (BLI) have expanded detection into the deep brain, substrates still need to penetrate the blood brain barrier (BBB). In addition, the degree of multiplexity for BLI and fluorescent imaging in vivo has not yet reached the levels achieved by biochemical detection methods, such as antibody-, DNA hybridization, or mass-spectrometry-based assays.

Unsurprisingly then, most brain-wide gene expression studies are performed on post-mortem brains, which involve tissue-destructive biochemical techniques (e.g., high-throughput RNA sequencing) that preclude longitudinal assessments of the same animal. To date, no technology has been developed for monitoring brain gene expression that is (1) noninvasive, (2) sensitive enough to measure expression in a small number of cells anywhere in the brain, (3) repeatable in the same animal, (4) capable of simultaneously imaging multiple molecule types, and (5) inexpensive and accessible to many research laboratories.

### BRIEF DESCRIPTION

Noninvasive efforts to map brain gene expression have been hampered by low sensitivity and limited access to the brain. As discussed herein a platform is described that enables multiplexed, noninvasive, and site-specific monitoring of brain gene expression through a class of engineered reporters, referred to herein as Released Markers of Activity (RMAs). Instead of detecting gene expression in the less accessible brain, RMA reporters exit the brain into the blood, where they can be easily measured with biochemical techniques. Expressing RMAs at a single brain site, typically covering ˜1% of the brain volume, provides up to a 39,000-fold signal increase over the baseline in vivo. Further, expression of RMAs in as few as several hundred neurons is sufficient for their reliable detection. When placed under a promoter upregulated by neuronal activity, RMAs may be used to measure neuronal activity in specific brain regions with a simple blood draw. During studies, it was observed that chemogenetic activation of cells expressing Fos-responsive RMA increased serum levels of RMA over 6-fold compared to non-activated controls. By contrast, a control RMA expressed under a constitutive neuronal promoter did not show such upregulation, demonstrating multiplexed ratiometric measurement with RMAs and proving specificity of neuronal activity discrimination. As discussed herein, the present approaches provide a noninvasive paradigm for repeatable and multiplexed monitoring of gene expression in an intact brain with sensitivity that is currently unavailable through other noninvasive gene expression reporter systems.

In one embodiment, a gene expression reporter is provided. In accordance with this embodiment, the gene expression reporter comprises: a released marker of activity (RMA) configured to cross a neural cell membrane and a blood brain barrier to report a gene expression in a region of a brain. In one such embodiment, the RMA comprises: a cell secretion signaling sequence; a detectable marker; and a fragment crystallizable-region (Fc-region) of an antibody.

In a further embodiment, a method for noninvasive, site-specific monitoring of expression of a gene is provided. In accordance with this method, one or more synthetic released markers of activity (RMAs) are expressed at a targeted brain site of a subject. Each RMA comprises: a cell secretion signaling sequence; a detectable marker; and a fragment crystallizable-region (Fc-region) of an antibody. Each RMA is configured to cross neuronal cell membranes; and cross a blood brain barrier of the subject. A blood sample of the subject is acquired. A detection assay is performed on the blood sample to detect and quantify presence of RMAs in the sample.

In an additional embodiment, a signal detection system is provided. In accordance with this embodiment, the signal detection system comprises an assay or detection technique configured to detect a released marker of activity (RMA) present in a blood sample. The RMA comprises: a cell secretion signaling sequence; a detectable marker; and a fragment crystallizable-region (Fc-region) of an antibody. The presence or quantity of RMA in the blood sample corresponds to expression of a gene in a targeted region of a brain.

### BRIEF DESCRIPTION OF THE DRAWINGS

In the drawings, like reference characters generally refer to like parts throughout the different views. Also, the drawings are not necessarily to scale, with an emphasis instead generally being placed upon illustrating the principles of the technology disclosed. In the following description, various implementations of the technology disclosed are described with reference to the following drawings, in which.

FIG. 1 schematically depicts an RMA workflow, in accordance with aspects of the present disclosure;

FIG. 2 schematically depicts an RMA reporter, in accordance with aspects of the present disclosure;

FIG. 3 schematically depicts RMA reporter passage through the blood brain barrier, in accordance with aspects of the present disclosure;

FIG. 4 depicts a schematic of RMA secretion in vitro for crossing the first barrier of the cellular membrane in a PC-12 culture, in accordance with aspects of the present disclosure;

FIG. 5 depicts the PC-12 culture of FIG. 4 d in a process flow of an experimental scheme for detecting secreted RMA using the PC-12 culture, in accordance with aspects of the present disclosure;

FIG. 6 depicts a graph of RMA secretion by PC-12 cells over time, in accordance with aspects of the present disclosure;

FIG. 7 depicts a series of graphs illustrating that replacing the secretion signal peptide of native Gluc with that of the murine antibody (IgK) preserves secretory function, in accordance with aspects of the present disclosure;

FIG. 8 depicts RMA secretion by astrocytes, in accordance with aspects of the present disclosure;

FIG. 9 depicts representative images of astrocytes stained 120 h post-transfection, in accordance with aspects of the present disclosure;

FIG. 10 depicts an example of a workflow for estimating the number of RMA secreted per PC-12 cell, in accordance with aspects of the present disclosure;

FIG. 11 depicts the estimated amount of RMA proteins released per PC-12 cell after 72 hr post-transfection, in accordance with aspects of the present disclosure;

FIG. 12 depicts an experimental scheme for testing reverse transcytosis of RMA for crossing the BBB into the bloodstream, in accordance with aspects of the present disclosure;

FIG. 13 graphically depicts plasma concentration of RMAs measured from collected blood after bilaterally injecting RMA proteins into the caudate putamen (CP) of the mice brains, in accordance with aspects of the present disclosure;

FIG. 14 depicts representative images of stained mice brain slices showing the remaining RMA proteins in the brain after 24 hr post-injection, in accordance with aspects of the present disclosure;

FIG. 15 depicts brain images of mice intracranially injected with RMA proteins, in accordance with aspects of the present disclosure;

FIG. 16 depicts concentrations of Gluc, Gluc-human IgG1 mFc, and Gluc-mouse IgG1 Fc (Gluc-RMA) in wild-type (WT) mice as a function of time after intravenous (i.v.) administration of each protein, in accordance with aspects of the present disclosure;

FIG. 17 depicts an example workflow for detecting brain gene expression in vivo using Gluc-RMA, in accordance with aspects of the present disclosure;

FIG. 18 illustrates bilateral injection sites for delivery of AAV encoding Gluc-RMA into the CP, in accordance with aspects of the present disclosure;

FIG. 19 depicts plasma bioluminescence signal and representative brain images showing the Gluc-RMA gene expression are illustrated along with a bar graph depicting RLU measured from collected blood samples, in accordance with aspects of the present disclosure;

FIGS. 20A, 20B, and 20C depict regional dependencies of Gluc-RMA by singly injecting the CP, CA1, and substantia nigra (SN) regions located in the striatum, hippocampus, and midbrain, respectively, in accordance with aspects of the present disclosure;

FIGS. 21A, 21B, and 21C illustrate plasma bioluminescence signal and representative images showing gene expression of Gluc-RMA in the target brain region-CP, CA1, and SN, respectively, along with respective bar graphs conveying RLU measured from the collected blood containing Gluc-RMA, in accordance with aspects of the present disclosure;

FIG. 22 illustrates a unilateral injection site for delivery of AAV encoding Gluc-RMA into the CP, in accordance with aspects of the present disclosure;

FIG. 23 depicts plasma bioluminescence signal and representative brain images showing the Gluc-RMA gene expression are illustrated along with a bar graph depicting RLU measured from the collected blood samples, in accordance with aspects of the present disclosure;

FIG. 24 depicts the relationship between the estimated number of transduced neurons and the plasma bioluminescence signals, in accordance with aspects of the present disclosure;

FIG. 25 depicts the relationship between the AAV dose to the plasma bioluminescence signals, in accordance with aspects of the present disclosure;

FIG. 26 depicts a schematic of injection sites in the CP and doses used for AAVs encoding Gluc-RMA-IRES-GFP (left hemisphere) or GFP (right hemisphere), in accordance with aspects of the present disclosure;

FIG. 27 depicts plasma bioluminescence signal and representative coronal views of the brain in the CP region showing the local expression of GFP at each relevant AAV dose, in accordance with aspects of the present disclosure;

FIG. 28 depicts images showing the expression of indicated genes including inflammatory markers in the left (Gluc-RMA) and right (GFP) hemispheres of CP at the three tested AAV doses, in accordance with aspects of the present disclosure;

FIG. 29 depicts a schematic workflow of selective expression of Gluc-RMA and GFP in the brain cells that express Cre recombinase, in accordance with aspects of the present disclosure;

FIG. 30 depicts plasma bioluminescence signal measured from collected blood samples containing the released Gluc-RMA after injection of AAVs, in accordance with aspects of the present disclosure;

FIG. 31 depicts representative imagery of a stained brain slice showing expression of Gluc-RMA and GFP among TH positive brain cells, in accordance with aspects of the present disclosure;

FIG. 32 schematically depicts a workflow for injection of AAVs encoding double-floxed Gluc-RMA and GFP at the CA1 region of the hippocampus and their selective expression in PV-positive interneurons, in accordance with aspects of the present disclosure;

FIG. 33 depicts plasma bioluminescence signal and a representative image of the stained brain slice showing the expression of Gluc-RMA and GFP among PV positive interneurons, in accordance with aspects of the present disclosure;

FIG. 34 depicts enlarged views of CA1, corresponding to region 1 (ipsilateral) and region 2 (contralateral) of the rectangular boxes shown in FIG. 33;

FIG. 35 depicts images stained to evaluate inflammation, in accordance with aspects of the present disclosure;

FIG. 36 depicts a bar graph illustrating plasma bioluminescence signal obtained with reduced AAV dose, in accordance with aspects of the present disclosure;

FIG. 37 depicts an experimental scheme in which PC-12 cells are transfected with plasmids encoding Gluc-RMA controlled under the Fos promoter, stimulated by NGF, and analyzed by luciferase assay for the secreted Gluc-RMA, in accordance with aspects of the present disclosure;

FIG. 38 depicts representative images of PC-12 stained 48 hr after the addition of media with or without NGF, in accordance with aspects of the present disclosure;

FIG. 39 depicts, via graphs, the percentage of PC-12 cells that express Gluc-RMA (left) or Fos (right), in accordance with aspects of the present disclosure;

FIG. 40 depicts results for RLU measured from culture media that contains the released Gluc-RMA, in accordance with aspects of the present disclosure;

FIG. 41 depicts an experimental scheme for detecting neuronal activity through blood tests with RMAs, in accordance with aspects of the present disclosure;

FIG. 42 depicts a schematic of the RAM system for detecting neuronal activity, in accordance with aspects of the present disclosure;

FIG. 43 depicts plasma bioluminescence signal of Gluc-RMA before (0 h) and after (24 h and 48 h) inducing chemogenetic activation at CP of the striatum with varying doses of CNO, in accordance with aspects of the present disclosure;

FIG. 44 illustrates average and individual mice plasma RLU values measured before and 2 h after i.p. injection, in accordance with aspects of the present disclosure;

FIG. 45 illustrates average plasma RLU values calculated by those of individual mouse measured after i.p. injection for those chemogenetically activated at the left CP, in accordance with aspects of the present disclosure;

FIG. 46 depicts representative images of stained brain slices of mouse perfused after 2 hr post-injection, in accordance with aspects of the present disclosure;

FIG. 47 depicts representative images of stained brain slices of mouse perfused after 48 hr post-injection, in accordance with aspects of the present disclosure;

FIG. 48 depicts quantification of cells expressing c-Fos and GFP at 2 hr post-CNO injection analyzed using image groups represented in FIG. 46, in accordance with aspects of the present disclosure;

FIG. 49 depicts quantification of cells expressing c-Fos and GFP at 48 hr post-CNO injection analyzed using image groups represented in FIG. 47, in accordance with aspects of the present disclosure;

FIG. 50 depicts the correlation between the counted number of GFP positive cells and the corresponding plasma bioluminescence signal of each mouse at 48 hr post-injection of either vehicle or CNO, in accordance with aspects of the present disclosure;

FIG. 51 depicts plasma RLU values presented analyzed in a similar format as shown with respect to FIG. 45, but with results obtained by conducting the study at the left hippocampus, in accordance with aspects of the present disclosure;

FIG. 52 depicts representative images of stained brain slices showing hippocampus of mouse perfused after 48 hr post-CNO injection, in accordance with aspects of the present disclosure;

FIG. 53 depicts graphs illustrating quantification of cells expressing c-Fos and GFP analyzed using image groups represented in FIG. 52, in accordance with aspects of the present disclosure;

FIG. 54 illustrates the plasma bioluminescence signal of Gluc-RMA normalized to Cluc-RMA upon chemogenetic activation in the left hippocampus, in accordance with aspects of the present disclosure;

FIG. 55 depicts a schematic of delivery of the BBB-permeable PHP.eB virus encoding Gluc-RMA to the brain through intravenous (i.v.) injection and subsequent collection of the blood or BLI for measurement of the brain gene expression, in accordance with aspects of the present disclosure;

FIG. 56 depicts Gluc-RMA expression in the brain, heart, kidney, and liver at 5 weeks post-injection, in accordance with aspects of the present disclosure;

FIG. 57 illustrates bioluminescence imaging of mice taken after 3 weeks post-PHP.eB delivery, in accordance with aspects of the present disclosure;

FIG. 58 depicts plasma RLU measured at 3-week post-PHP.eB delivery, in accordance with aspects of the present disclosure;

FIG. 59 illustrates the PHP.eB dose response to the plasma bioluminescence signal, in accordance with aspects of the present disclosure;

FIG. 60 depicts images of Gluc-RMA expression in the striatum, cortex, midbrain, and hippocampus in the brain of mice administered with different PHP.eB doses, in accordance with aspects of the present disclosure;

FIG. 61 depicts a gating strategy for estimating the percentage of GFP positive cells, in accordance with aspects of the present disclosure; and

FIG. 62 illustrates the percentage of PC-12 cells expressing GFP analyzed by FACS, in accordance with aspects of the present disclosure.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and enterprise-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present disclosure, the articles “a,” “an,” and “the” are intended to mean that there are one or more of the elements. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features.

Discussed herein are a class of genetically-encoded reporters of gene expression that are excreted into the interstitial space of the brain and may then be transported into the blood. As used herein, such gene expression reporters are referred to as Released Markers of Activity (RMAs). As described herein, RMAs are proteins that contain a cell secretion signaling sequence, a detectable marker (e.g., luciferase, fluorescent protein, an epitope of an antibody, or other suitable detectable marker), and an Fc-region of an antibody that enables reverse transcytosis across the blood brain barrier (BBB).

Once in the blood, and as discussed in greater detail below, the RMAs can be detected with a suitable biochemical serum analysis technique without the confounds or issues associated with imaging within solid tissues. These gene expression reporters are suitable for noninvasive, sensitive, site-specific, and repeatable measurement of gene expression in an intact brain. In particular, RMAs leverage two phenomena-first, secretion from the neuron to release RMAs into the interstitial space and, second, reverse transcytosis to allow RMAs to cross the BBB into the bloodstream. Because RMAs enter the blood, they can be detected using a suitable, sensitive biochemical technique. Moreover, RMAs are compatible with multiplexed biochemical detection assays, some of which can reach single-molecule sensitivity. Unlike other methodologies that measure the concentration of reporters within the brain, RMAs present an approach to monitoring brain gene expression that can be achieved with a simple blood draw.

In presenting and describing the present techniques, reference is made to various studies performed as part of developing and testing these techniques. By way of example, in one study, RMAs were expressed in multiple brain regions, including the striatum, hippocampus, and midbrain, and the reporters were detectable after a single viral injection. In particular, RMAs were measured at high levels in the plasma and were measurable even in the sub-thousand neuron range. Further, chemogenetic activation of specific brain regions were observed to lead to an increase in RMA signals without significant change in constitutive gene expression, demonstrating that RMAs can be used to discriminate neuronal activity in vivo. With this in mind, and as discussed herein, techniques employing RMAs appear to be suitable for noninvasive monitoring of gene expression dynamics in the brain.

The following discussion is broken into discrete sections or subsections to help convey and explain particular aspects of the present techniques deemed of interest. Further aspects related to methodology employed in the described studies are included after so as to simplify the description while providing additional details for those in need of such supplemental material. With this in mind, certain aspects of the present techniques that are related to the use of RMA reports are described in greater detail.

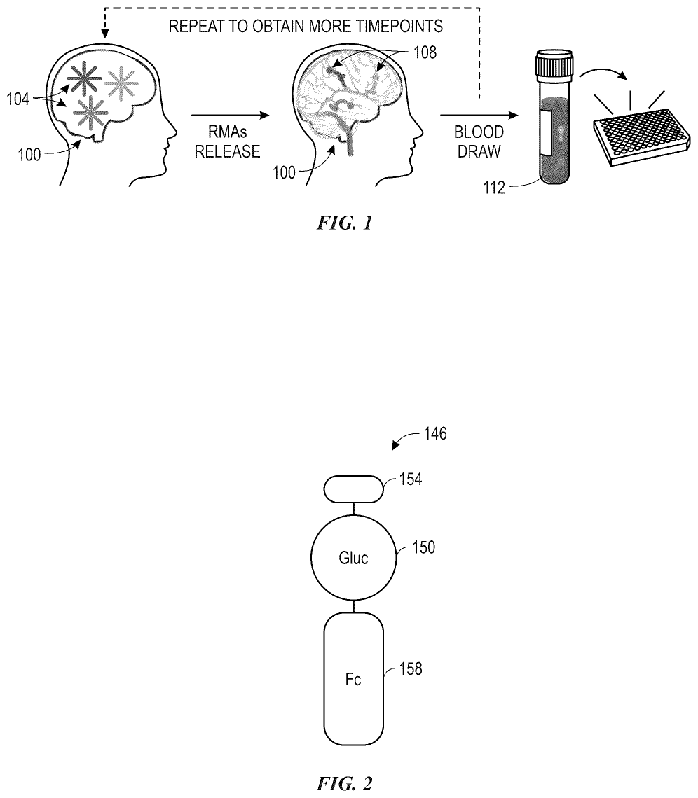

Engineering the RMA reporter as a serum marker for gene expression in the brain—The RMA platform as described herein involves genetically labelling targeted brain sites with synthetic blood brain barrier (BBB)-permeable RMA reporters, allowing for their release into the blood, and subsequently measuring the level of plasma RMAs to quantify gene expression in the brain. This is illustrated graphically in FIG. 1, in which RMAs are illustrated as being expressed (leftmost image) in specific regions (expression regions 104) of a subject's brain 100. Subsequently, the RMAs 108 are released via reverse transcytosis into the blood of the subject. As may be appreciated, for an RMA to be transported from the brain to the blood, it must cross two biological barriers: the neuronal cell membrane and the BBB. With this in mind, RMA reporters as discussed are designed so as to contain two functional protein domains, one to facilitate crossing the cell membrane and another for crossing the BBB. In the depicted example, a blood draw or other blood sampling technique may be performed and the blood sample 112 used as the basis for a pooled biochemical detection of the released RMAs (rightmost image). As shown in FIG. 1, these steps may be repeated at different points in time (e.g., a longitudinal study) to obtain additional useful data, such as to observe trends or differences attributable to the different timepoints sampled.

Turning to FIG. 2, and as noted above, the presently described RMA reporters 146 are designed so as to include two functional protein domains, one to facilitate crossing the cell membrane and another for crossing the BBB. With respect to these functional protein domains and their relevance, it may be appreciated that to perform the functions described herein, the RMAs 146 first undergo exocytosis to move from a respective neuron into the extracellular space. In one example, and as shown in FIG. 2, to enable crossing the cell membrane, Gaussia luciferase (Gluc), a highly sensitive reporter that can also be used with bioluminescence imaging (BLI) techniques, was selected to be incorporated in the RMA design as a detectable protein marker 150. In this manner the RMA 146 is endowed with both a cell secretion sequence 154 and a detectable protein marker domain 150.

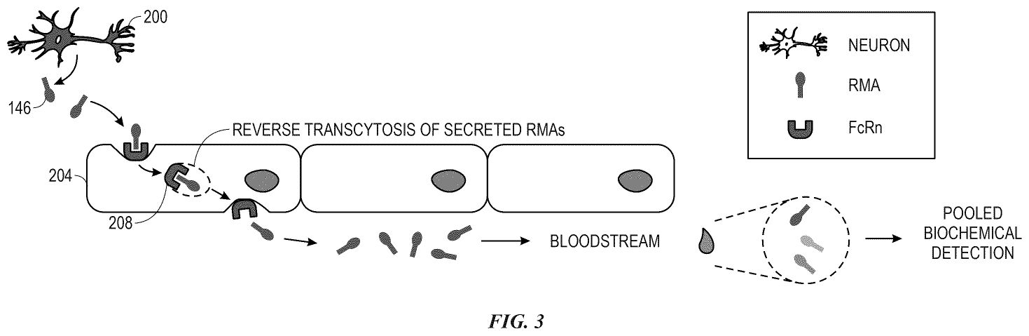

In addition, to enable RMAs 146 to traverse the BBB, a feature of reverse transcytosis was leveraged that mediates the efflux of antibodies from the central nervous system (CNS) back into systemic circulation. In this mechanism, the fragment crystallizable (Fc) region 158 of an antibody binds to the neonatal Fc-receptor (FcRn) expressed on the BBB in a pH-dependent manner. Fc binds avidly to FcRn at the endosomal pH (<6.5) in the CNS space but not at the physiological pH (7.4) in the blood. The strict pH-dependent binding of Fc to FcRn thus, in effect, favors the unidirectional release of antibodies from the brain into the blood. Aspects of this process are illustrated in FIG. 3, in which the neurons are denoted by reference number 200, RMAs by reference number 146, neonatal Fc receptor (FcRn) by reference number 208, and the blood brain barrier (BBB) by reference number 204. In this example, an RMA 146 is expressed in transduced cells (e.g., neuron 200) and secreted into the surrounding tissue. RMAs 146 are fused to a moiety (Fc 158 of FIG. 2) that recognizes neonatal FcRn 208, which mediates the transport of RMAs 146 into the blood. The process of transport is called reverse transcytosis. Once in the blood, all RMAs 146 in the sample can be detected using any biochemical method. To functionalize RMAs 146 to undergo reverse transcytosis, Fc regions 158 were selected of three different immunoglobulin G (IgG) antibodies: the human IgG1 monomeric Fc (mFc) and mouse IgG1 and IgG2a Fcs. Each Fc was respectively fused to the Gluc reporter 150, in one example, to construct Gluc-Fc RMA variants.

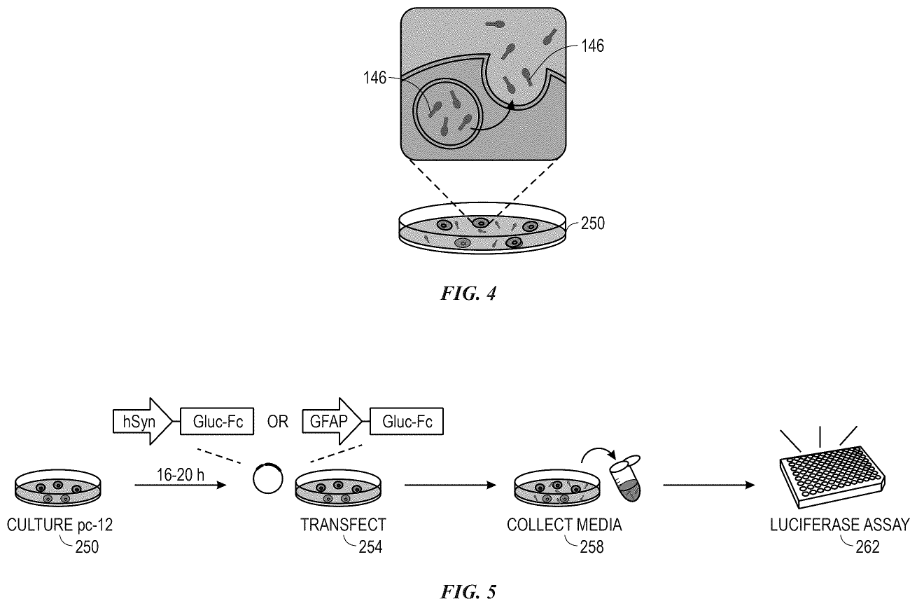

Secretion of RMAs in vitro—To assess RMA secretion from cells, the Gluc-Fc variants were expressed under the neuron-specific hSyn promoter in PC-12, a widely-used murine cell line for studying neurosecretion. Subsequently, the amount of secreted Gluc-Fc in the culture media was measured by luciferase assay. Aspects of this approach are illustrated in FIGS. 4 and 5. In FIG. 4 a schematic of RMA 146 secretion in vitro is illustrated for crossing the first barrier of the cellular membrane in a PC-12 culture 250. Turning to FIG. 5, the PC-12 culture of FIG. 4 is illustrated in a process flow of an experimental scheme for detecting secreted RMA using the PC-12 culture. The depicted experimental schema includes a 16-20 hr incubation of the PC-12 culture followed by a transfection step 254, media collection 258 and a luciferase assay 262.

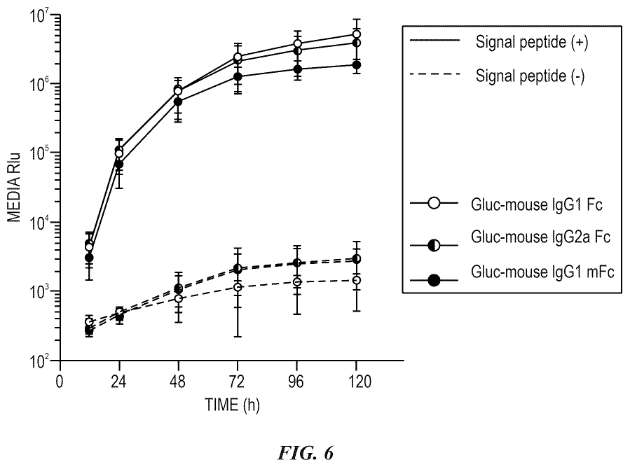

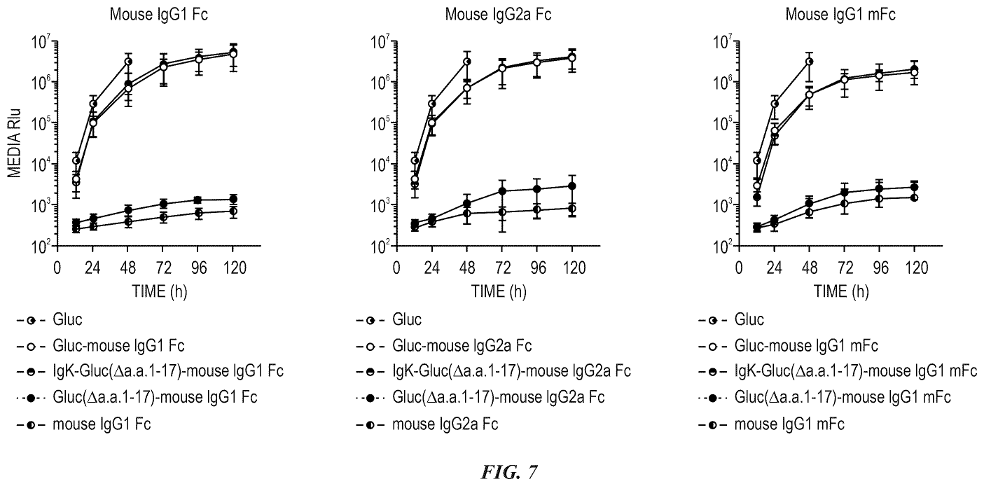

As depicted in the example, for each variant, a truncation mutant (Gluc-Fc Δ a.a. 1-17), which lacks the N-terminal secretion signal peptide was also tested. Observed results showed that all Gluc-Fc variants with the signal peptide accumulated in the media over time, indicating that fusing Gluc to Fc does not compromise its ability to be secreted. This is illustrated in FIG. 6 and FIG. 7. Turning to FIG. 6 a graph of RMA secretion by PC-12 cells over time is provided. Relative luminescence unit (RLU) values measured from the culture media reveal the signal peptide-dependent secretion of Gluc-Fc RMA variants. n=5 independent cultures analyzed. Turning to FIG. 7, signal-peptide dependency of RMA secretion is illustrated for the three variants tested. RMAs 146 lacking the signal peptide are truncated by the first 17 amino acids of Gluc (Δa.a.1-17). As shown, replacement of the native signal-peptide of Gluc with the murine Ig Kappa (IgK) did not alter the secretion efficiency. n=5 independent cultures analyzed.

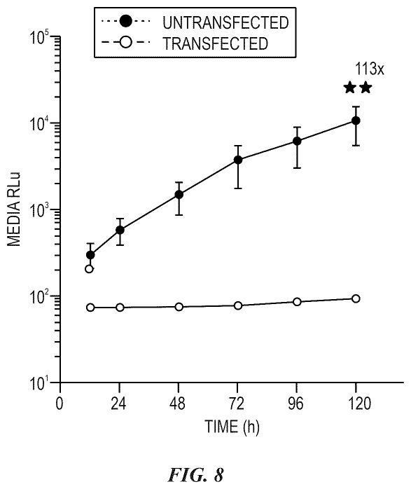

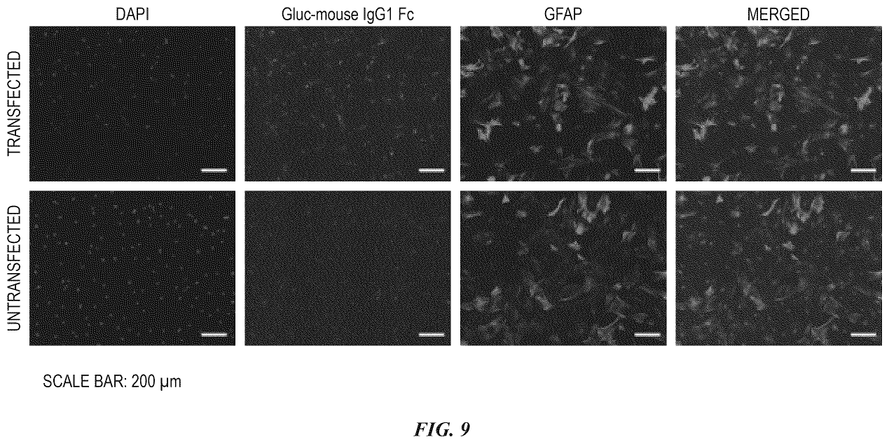

In addition, as shown in FIG. 7 it was observed that replacing the secretion signal peptide of native Gluc with that of the murine antibody (IgK) preserves secretory function, suggesting that the secretion signal peptide is an interchangeable but essential component of efficient RMA 146 release from the cells. To examine whether Gluc-Fc can be secreted from non-neuronal cells, such as glial cells, the astrocyte-specific GFAP promoter was utilized to express Gluc-Fc in rat primary astrocytes, as shown in FIG. 5. Gluc-Fc was secreted stably into the medium, yielding luciferase signals that were 113-fold higher than that of the untransfected control at 5 days post-transfection. This result is illustrated in FIGS. 8 and 9, in which FIG. 8 depicts RMA secretion by astrocytes. At 120 hr, the bioluminescence signals for the transfected cells were 113-fold higher than those for the untransfected cells. Gluc-mouse IgG1 Fc was selected as the test RMA. n=5 independent cultures were analyzed, **P<0.01, using unpaired two-tailed t-test. Turning to FIG. 9, representative images of astrocytes stained 120 h post-transfection are depicted.

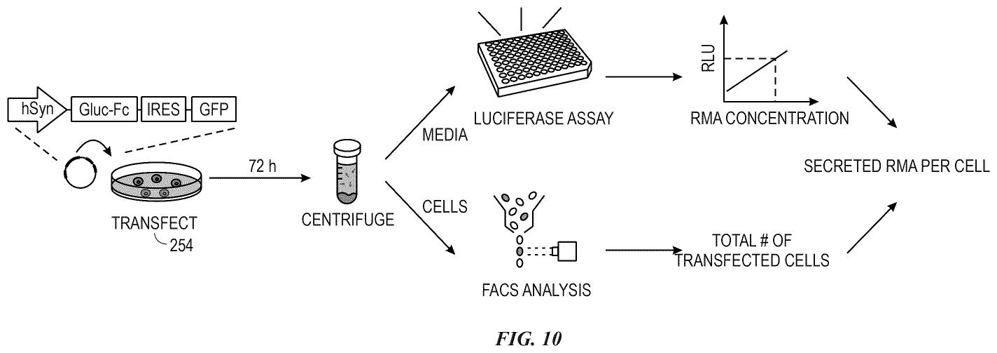

Given the attomolar detection sensitivity of Gluc, the secretion rates of the Gluc-Fc variants were studied to estimate the minimum number of cells required for detection using luciferase assay. Gluc-Fc and GFP were bicistronically-expressed under the hSyn promoter in PC-12 cells and the amount of Gluc-Fc released into the media was quantified. The amount of Gluc-Fc per cell were normalized using the total number of GFP-positive cells. An example of this workflow for estimating the number of RMA secreted per PC-12 cell is illustrated in FIG. 10. In accordance with this example workflow, delivering a bicistronic vector co-expressing Gluc-Fc and GFP allows for measurement of the secreted RMAs and the number of transfected cells to calculate the average amount of RMA proteins released per cell. In the depicted workflow, IRES equates to Internal ribosome entry site and FACS equates to Fluorescence-activated cell sorting. Data are shown as mean±SD.

On average, Gluc showed the highest secretion rate with 49.2±12.8 amol per cell, followed by Gluc-mouse IgG2a Fc (31.6±12.2), Gluc-mouse IgG1 Fc (30.1±6.8), and Gluc-human IgG1 mFc (7.6±1.2). These results are illustrated graphically in FIG. 11, which depicts the estimated amount of RMA proteins released per PC-12 cell after 72 hr post-transfection. ****P<0.0001, ns (not significant) in comparison of each variant with and without the signal peptide, using Two-way ANOVA, Sidak's test. These secretion rates suggest that one PC-12 cell could be sufficient for readout and demonstrate detectability of secreted RMAs 146 in vitro. RMAs lacking the signal peptide showed no measurable secretion.

RMAs exit from the brain into the blood—To examine whether RMAs can cross the BBB, a study was conducted in which direct injections of RMA proteins were performed into the caudate putamen (CP) of the mouse brain, where IgG efflux is mediated by the interaction between Fc and FcRn. Among the RMA variants, Gluc-mouse IgG1 Fc was selected, herein referred to as Gluc-RMA, as it contains the Fc of the native host. The protein sequence of the embodiment of Gluc-RMA described herein is as follows:

(SEQ ID NO. 1)
MGVKVLFALICIAVAEAKPTENNEDFNIVAVASNFATTDLDADRGK
LPGEKLPLEVLKELEANARKAGCTRGCLICLSHIKCTPKMKKFIP
GRCHTYEGDKESAQGGIGEAIDDIPEIPGFKDLEPIEQFIAQVDL
CVDCTTGCLKGLANVQCSDLLKKWLPQRCATFASKIQGQVDKIKG
AGDDGSVPEVSSVFIFPPKPKDVLTITLTPKVTCVVVDISKDDPE
VQFSWFVDDVEVHTAQTQPREEQFNSTFRSVSELPIMHQDWLNGK
EFKCRVNSAAFPAPIEKTISKTKGRPKAPQVYTIPPPKEQMAKDK
VSLTCMITDFFPEDITVEWQWNGQPAENYKNTQPIMDTDGSYFVY
SKLNVQKSNWEAGNTFTCSVLHEGLHNHHTEKSLSHSPGK

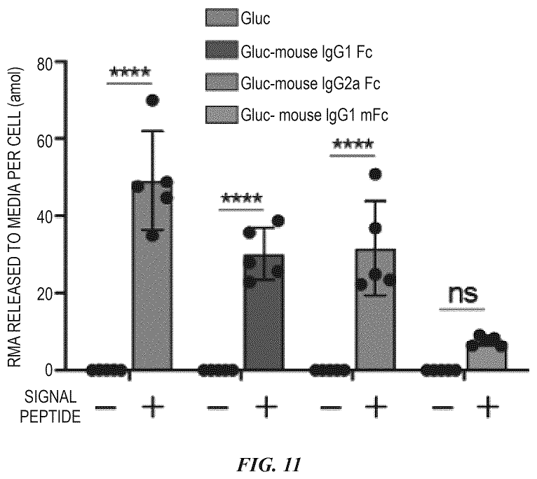

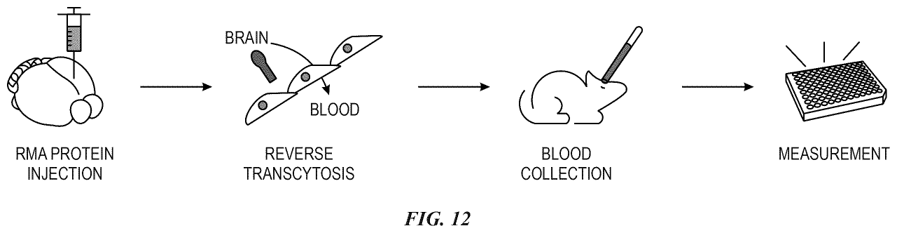

As controls, Gluc was selected, which does not contain the Fc domain, and Gluc-RMA (I203A+H260A+H385A), which carries mutations in the Fc region that abolish FcRn binding. 20 pmol of Gluc, Gluc-RMA (I203A+H260A+H385A), or Gluc-RMA were injected into each hemisphere and the released reporters measured in blood samples. The respective work flow is illustrated in FIG. 12, which depicts the experimental scheme for testing reverse transcytosis of RMA for crossing the BBB into the bloodstream. Within 2 hr after the injection, a significant rise in the plasma concentration of Gluc-RMA was observed (24.4-fold and 43.7-fold higher than that of Gluc and Gluc-RMA (I203A+H260A+H385A), respectively), suggesting that RMA transportation from the brain to the blood occurs through an Fc-dependent mechanism.

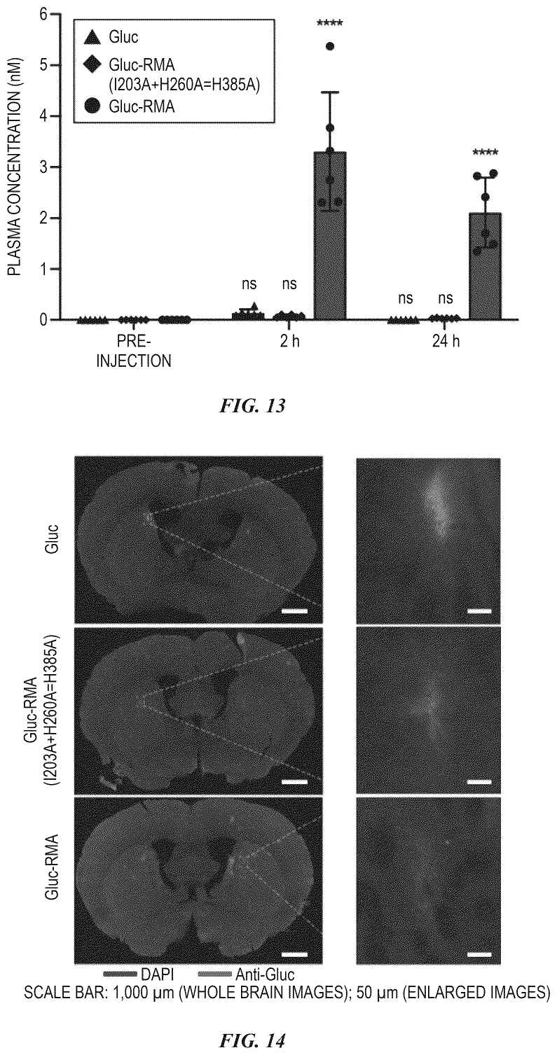

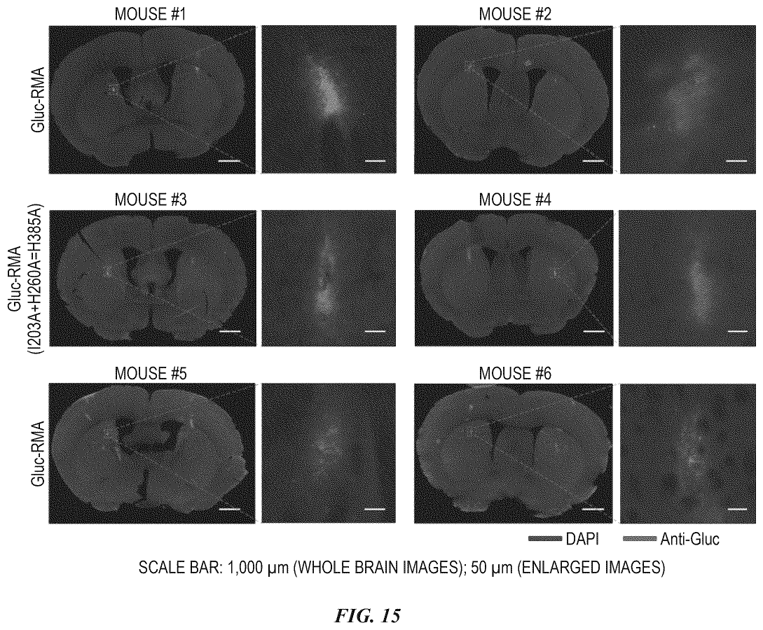

Aspects of these results are illustrated in FIGS. 13-15. FIG. 13 graphically depicts plasma concentration of RMAs 146 measured from the collected blood after bilaterally injecting 1 μL of 20 μM (20 μmol) RMA proteins per injection into CP of the mice brains. n=6 independent mice analyzed. ****P<0.0001, ns (not significant) in comparison with the plasma concentration of the respective RMA measured from the pre-injection time point, using two-way ANOVA Sidak's test. FIG. 14 depicts representative images of the stained mice brain slices showing the remaining RMA proteins in the brain after 24 hr post-injection. All data are shown as mean±SD. FIG. 15 depicts additional brain images of mice intracranially injected with RMA proteins. Images of the brain 24 hr after the injection of a) Gluc, b) Gluc-RMA (I203A+H260A+H385A), or c) Gluc-RMA proteins. Each image is obtained from an independent mouse.

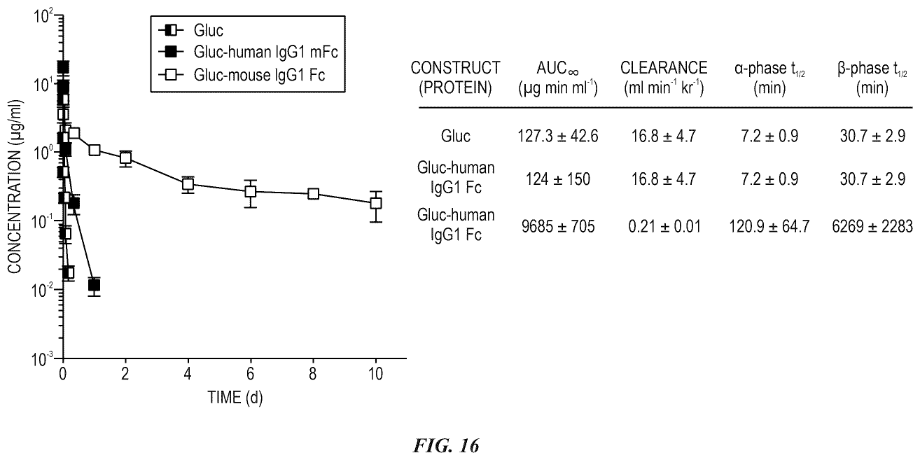

Between 2 and 24 hr, it was observed that Gluc-RMA plasma levels decreased only by 35±13%, whereas Gluc and Gluc-RMA (I203A+H260A+H385A) displayed a 96±2% and 53±17% reduction, respectively, close to the baseline, as shown in FIG. 13. It was observed that the B-phase half-life (t_{1/2}) of Gluc-RMA in the blood was substantially greater than that of Gluc (6,269±2,283 min vs 31±3 min). This is illustrated graphically in FIG. 16 in which serum half-life of Gluc-RMA is illustrated and where AUC equates to area under the curve. In particular, FIG. 16 depicts concentrations of Gluc, Gluc-human IgG1 mFc, and Gluc-mouse IgG1 Fc (Gluc-RMA) in the wild-type (WT) mice as a function of time after intravenous (i.v.) administration of each protein with the dose of 2 mg/kg. A Tto-compartment elimination model was used to analyze the pharmacokinetic parameters. n=2 to 6 blood samples analyzed. Data are shown as mean±SD. These data are consistent with the pharmacokinetics of Fc-fusion proteins, wherein the fusion protein acquires extended fin through its interaction with FcRn, which helps to reduce protein degradation. Together, these data suggest that Gluc-RMA is released from the brain and circulates with multi-hour-long t_{1/2}, allowing for its accumulation in blood.

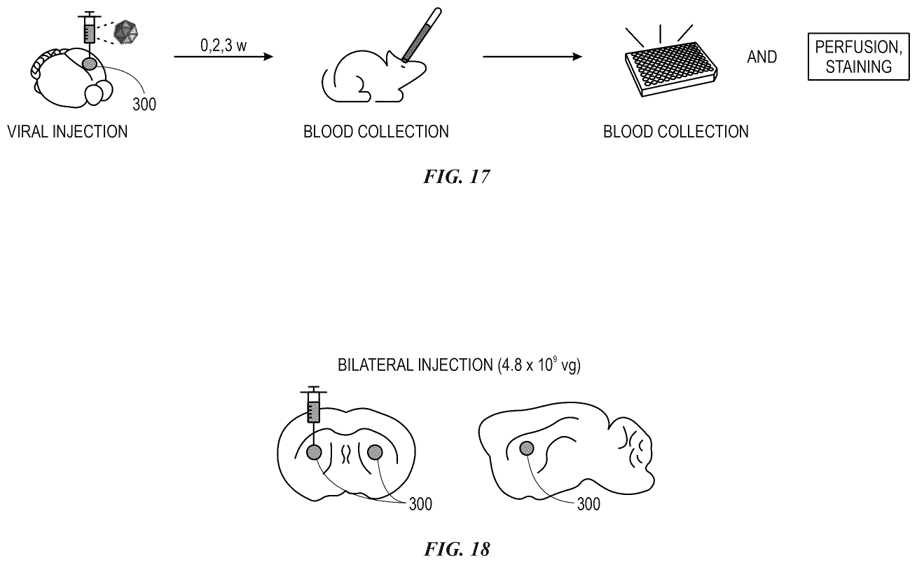

RMAs detect gene expression in as few as hundreds of neurons-After establishing that Gluc-RMA can traverse out of the brain and into the blood, a study was performed to determine Gluc-RMA could be used to detect brain gene expression in vivo. Adeno-associated virus (AAV) encoding both Gluc-RMA and GFP controlled under the constitutive neuronal hSyn promoter were injected into the mouse brain and the plasma assayed for the released reporter. An example of this workflow is depicted in FIG. 17. In this example, an experimental scheme is illustrated for detecting gene expression in brain regions. A mouse was injected with AAV encoding Gluc-RMA and subjected to blood collection for measurement of the released Gluc-RMA reporters. In particular, both hemispheres of the CP were both injected and respective 36,867- and 49,530-fold signal increases were found at 2 and 3 weeks post-delivery when compared with 0 weeks (baseline).

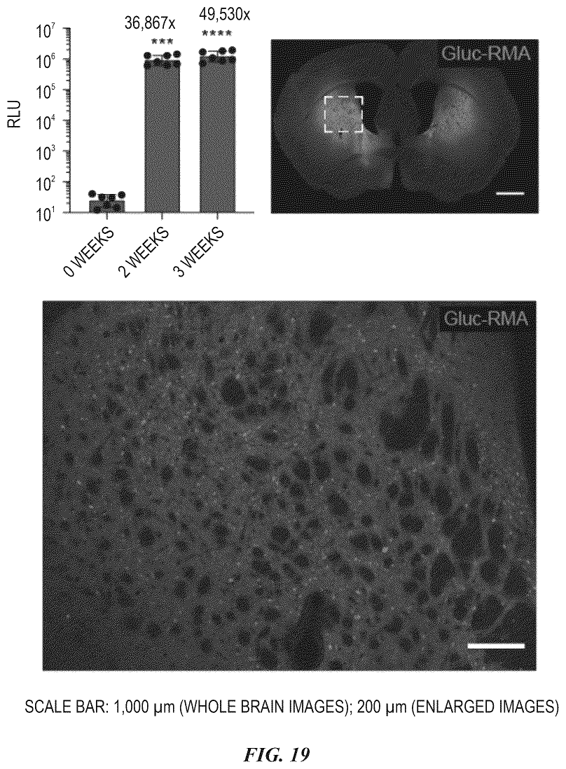

Aspects of this study and the results are illustrated in FIGS. 18 and 19, in which FIG. 18 illustrates the bilateral injection sites for delivery of AAV encoding Gluc-RMA into the CP. The depicted brain schemes display the coronal (left) and sagittal (right) views of the injection sites 300. The AAV dose indicates total viral genomes injected per mouse. Turning to FIG. 19, plasma bioluminescence signal and representative brain images showing the Gluc-RMA gene expression are illustrated along with a bar graph depicting RLU measured from the collected blood samples. The number above each bar of the bar graph indicates the signal fold increase compared with the signal at 0 weeks. Right and bottom images depict brain slices stained against Gluc-RMA after 3 weeks post-AAV injection. *P<0.05, ***P<0.001, ****P<0.0001, ns (not significant), in comparison with the plasma signal at 0 weeks, using one-way ANOVA, Tukey's test. Data are shown as mean±SD.

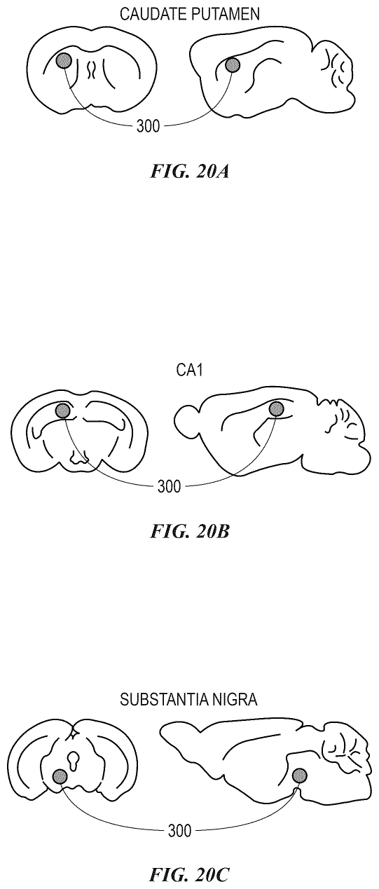

Given these high Gluc-RMA signals, further study and analysis was performed to examine possible regional dependencies of Gluc-RMA by singly injecting the CP, CA1, and substantia nigra regions located in the striatum, hippocampus, and midbrain, respectively, as illustrated in FIGS. 20A, 20B, and 20C. AAV injection sites 300 are shown in FIGS. 20A, 20B, and 20C, in which left and right brain schemes show the coronal and sagittal views, respectively. Injection sites 300 correspond to the target injection sites in the caudate putamen (CP, striatum) (FIG. 20A), CA1 (hippocampus) (FIG. 20B), and substantia nigra (SN, midbrain) (FIG. 20C.

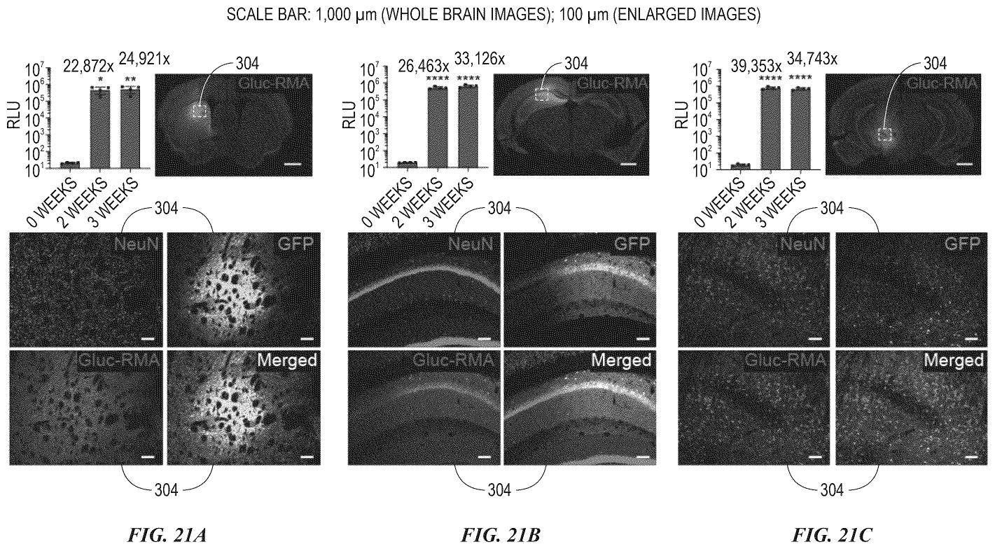

Per the observed results, >20,000-fold higher signals over baseline were observed in all three regions, demonstrating that gene expression in various local brain regions can lead to detectable RMA signal levels in the serum, regardless of their location. Furthermore, the signal levels persisted up to the 3^{rd} week, possibly due to plasma Gluc-RMA reaching steady-state, wherein the rate of production matches the rate of degradation under the constitutive expression. This is illustrated in FIGS. 21A, 21B, and 21C, illustrating plasma bioluminescence signal and representative images showing gene expression of Gluc-RMA in the target brain region-CP, CA1, and SN, respectively, along with respective bar graphs conveying RLU measured from the collected blood containing Gluc-RMA after injecting 2.4×10⁹ vg AAVs into the respective brain sites indicated in FIGS. 20A, 20B, and 20C, respectively. The number shown above each bar of the bar graphs refers to the signal fold increase compared with the 0 weeks baseline. n=4 independent mice analyzed. *P<0.05, **P<0.01, ****P<0.0001, in comparison with the signal at 0 weeks, using One-way ANOVA, Tukey's test. Data are shown as mean±SD. Illustrated whole-brain images illustrate via rectangular region (inset 304) Gluc-RMA expression at the local injected sites. Beneath, enlarged views of the inset 304 are provided.

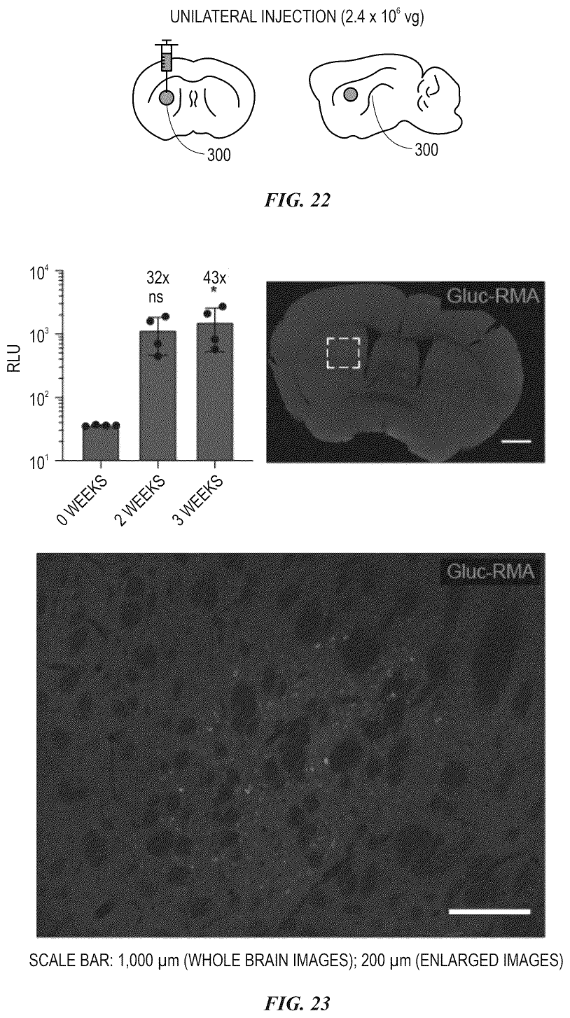

To determine the fewest number of neurons that can be transduced and later discerned in histological images, further study was performed using another injection into a single CP site using 1/1000^{th} of the initial AAV dose (see Methods subsection herein). A 43-fold signal increase was observed over the baseline in approximately 815 (±327, n=3) neurons as estimated using histological analysis, suggesting that Gluc-RMA reliably detects ˜0.001% of neurons in the mouse brain. These results are illustrated in FIGS. 22 and 23, in which FIG. 22 illustrates the unilateral injection site 300 for delivery of AAV encoding Gluc-RMA into the CP. The depicted brain schemes display the coronal (left) and sagittal (right) views of the injection sites (blue circles). The AAV dose indicates total viral genomes injected per mouse. Turning to FIG. 23, plasma bioluminescence signal and representative brain images showing the Gluc-RMA gene expression are illustrated along with a bar graph depicting RLU measured from the collected blood samples. The number above each bar of the bar graph indicates the signal fold increase compared with the signal at 0 weeks. Right and bottom images depict brain slices stained against Gluc-RMA after 3 weeks post-AAV injection. *P<0.05, ***P<0.001, ****P<0.0001, ns (not significant), in comparison with the plasma signal at 0 weeks, using one-way ANOVA, Tukey's test. Data are shown as mean±SD.

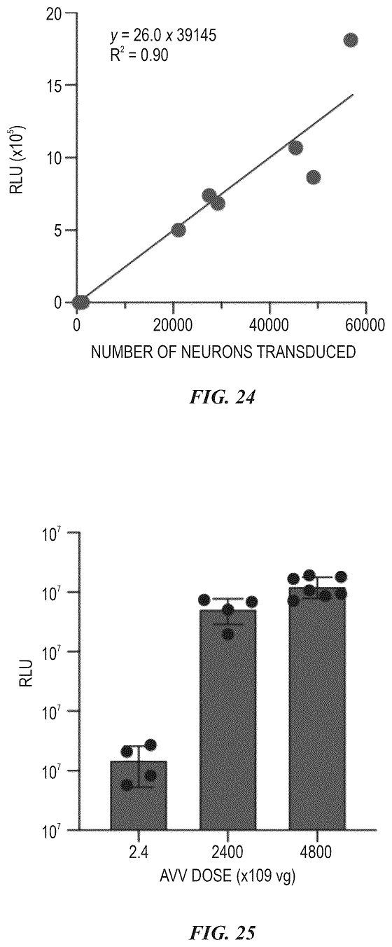

To test the correlation between plasma signals and the number of transduced neurons or AAV dose, data was collected and analyzed for injections in the CP with different AAV doses. A linear relationship (r²=0.90) was discovered for up to 56,841 transduced neurons (˜0.1% of mouse neurons). This is illustrated in FIGS. 24 and 25, in which the correlation between the estimated number of transduced neurons (FIG. 24) and the AAV dose to the plasma bioluminescence signals (FIG. 25) is illustrated. Taken together, these results indicate that Gluc-RMA provides a linear dynamic range throughout all tested conditions with a sensitivity to monitor gene expression in as few as hundreds of neurons.

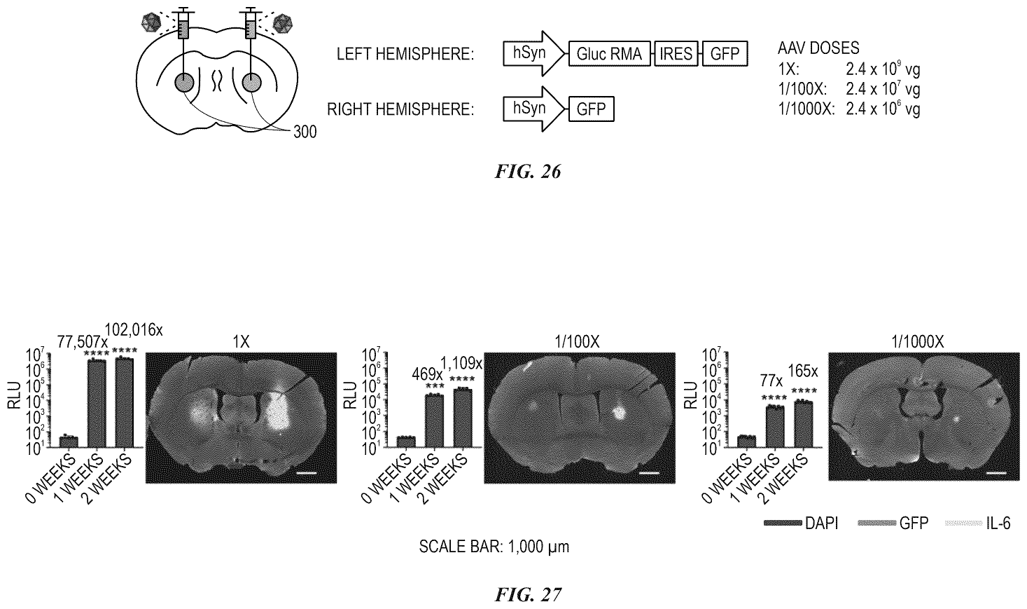

Inflammatory response of RMAs at varying AAV doses-FcRn interacts with the Fc domain of antibodies, thereby activating signaling pathways that are involved in both innate and adaptive immune responses, including the release of pro-inflammatory cytokines, promotion of phagocytosis, or mediation of autoimmune diseases. Since Gluc-RMAs contain both the Fc domain and Gluc from a foreign host, the inflammatory response in the brain induced by Gluc-RMA was examined and the safe AAV doses that minimize inflammation were identified. To accomplish this, Gluc-RMA and GFP were both expressed in CP at the left hemisphere and only GFP for comparison at the right hemisphere by injecting three different AAV doses (2.4×10⁹ vg (1×), 2.4×10⁷ vg (1/100×), and 2.4×10⁶ vg (1/1000×)). This experimental setup is illustrated for reference in FIGS. 26 and 27. In particular, FIG. 26 depicts a schematic of the injection sites 300 in CP and doses used for AAVs encoding Gluc-RMA-IRES-GFP (left hemisphere) or GFP (right hemisphere). Gluc-RMA-IRES-GFP co-expresses both Gluc-RMA and GFP, allowing for the assessment of inflammation caused by Gluc-RMA in addition to GFP expression. In FIG. 27 plasma bioluminescence signal and representative coronal views of the brain in the CP region are provided showing the local expression of GFP at each relevant AAV dose for n=5 to 7 independent mice analyzed. The number shown above each bar represents the signal fold increase compared with the 0 weeks baseline. ***P<0.001, ****P<0.0001, in comparison with the signal at 0 weeks, using One-way ANOVA, Tukey's test. Data are shown as mean±SD.

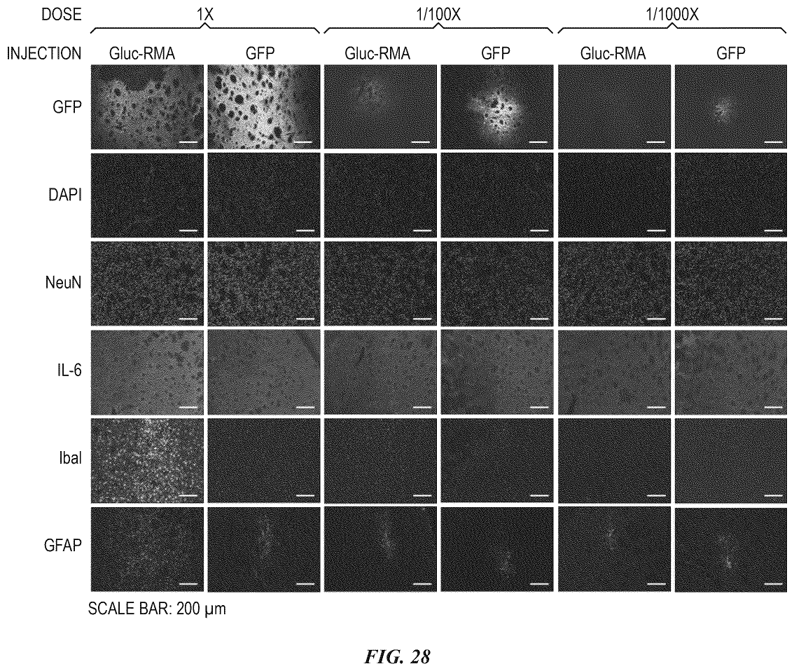

At 2 weeks post-delivery, the brains were assessed by staining for NeuN (neuronal loss), IL-6 (proinflammatory cytokine), IbaI (activation of microglia and phagocytosis), and GFAP (astrocyte activation and astrogliosis). It was observed that at 1× dose Gluc-RMA elicited significantly higher microglial (IbaI) and astrocyte (GFAP) response compared to GFP but not at 1/100× and 1/1000× doses, and no significant neuronal loss or increase in IL-6 was observed across all doses. Turning to FIG. 28, images are provided showing the expression of indicated genes including inflammatory markers in the left (Gluc-RMA) and right (GFP) hemispheres of CP at the three tested AAV doses. An individual brain slice was used to stain each inflammatory marker. On average, the plasma Gluc-RMA signals for doses of 1/100× and 1/1000× were over 1,000-fold and 100-fold higher, respectively, than the baseline (as illustrated in FIG. 27). The results indicate that even at lower doses, reliable detection of gene expression could be achieved as early as 2 weeks post-delivery with minimal immunogenicity, and that using the high 1× dose might be unnecessary or even excessive.

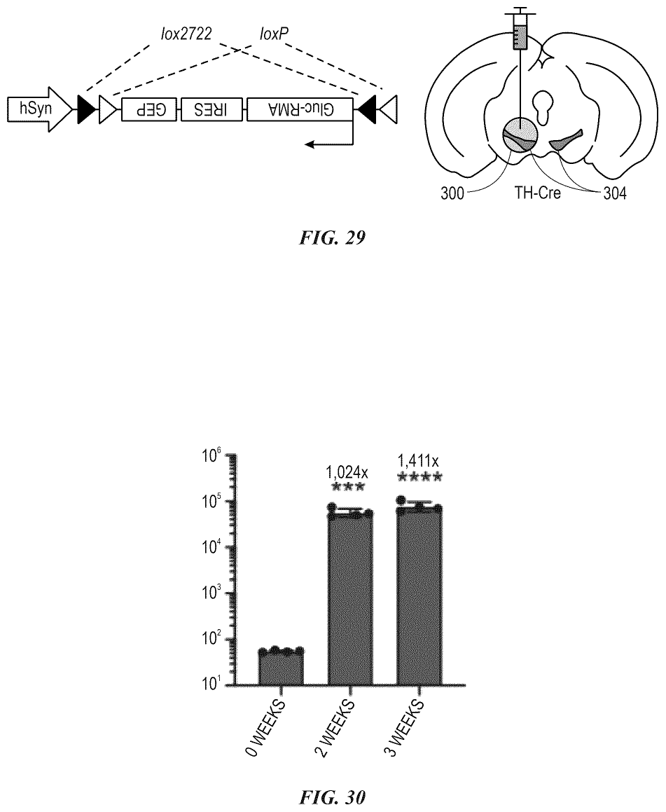

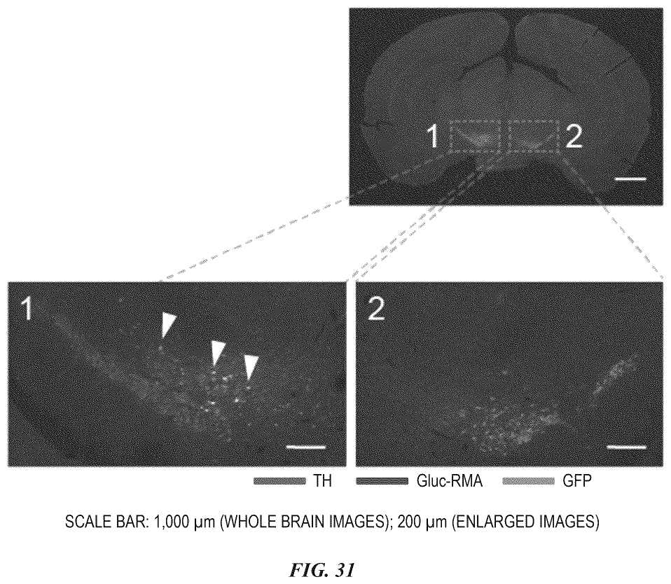

High-sensitivity monitoring of brain cell type-specific gene expression using Cre lines—To observe gene expression in a selected brain cell type, hSyn-controlled double-floxed Gluc-RMA-IRES-GFP was delivered into TH-Cre mice, which express Cre in dopaminergic neurons under the control of a tyrosine hydroxylase (TH) promoter. The left ventral tegmental area (VTA), which is rich with TH-positive cells, was specifically targeted. This workflow is illustrated in FIG. 29, illustrating a schematic of selective expression of Gluc-RMA and GFP in the brain cells that express Cre recombinase. Cre recognizes the double-floxed gene (Gluc-RMA-IRES-GFP) flanked by the two loxP sites and inverts the sequence back to the correct orientation, which results in the expression of Gluc-RMA and GFP. A coronal view is illustrated of the AAV injection site 300 at the VTA region of the brain that expresses Cre (regions 340) in TH-Cre mice. At 2 and 3 weeks post-delivery, plasma Gluc-RMA signals were 1,022- and 1,409-fold higher than at 0 weeks, respectively. This is illustrated in FIG. 30, which depicts plasma bioluminescence signal measured from the collected blood samples containing the released Gluc-RMA after injection of AAVs at the dose of 1.2×10⁹ vg. The number shown above each bar refers to the signal fold increase compared with the 0-week baseline. n=4 independent mice analyzed. ***P<0.001, ****P<0.0001, in comparison with the signal at 0 weeks, using One-way ANOVA, Tukey's test. Data are shown as mean±SD. Expressions of Gluc-RMA and GFP were specific to TH-positive cells at the local injection site, as shown in FIG. 31, providing evidence that Gluc-RMA can detect brain gene expression in small neuronal cell populations, and in a cell type-specific manner. In particular, FIG. 31 depicts representative imagery of a stained brain slice showing expression of Gluc-RMA and GFP among TH positive brain cells.

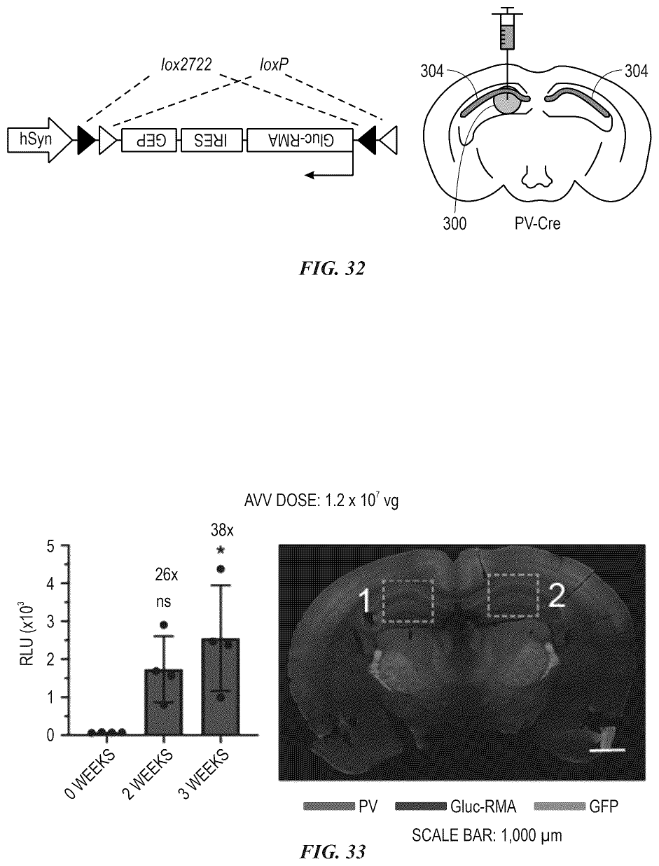

To further evaluate the sensitivity of the described system, lower doses of 1/100× and 1/1000× were tested on PV-Cre mice, which allowed expression of Gluc-RMA in sparsely distributed parvalbumin (PV)-positive interneurons within the CA1 of hippocampus. A depiction of this workflow is illustrated in FIG. 32, which schematically depicts injection of AA Vs encoding double-floxed Gluc-RMA and GFP at the CA1 region of the hippocampus (injection region 300) and their selective expression in PV-positive interneurons 308.

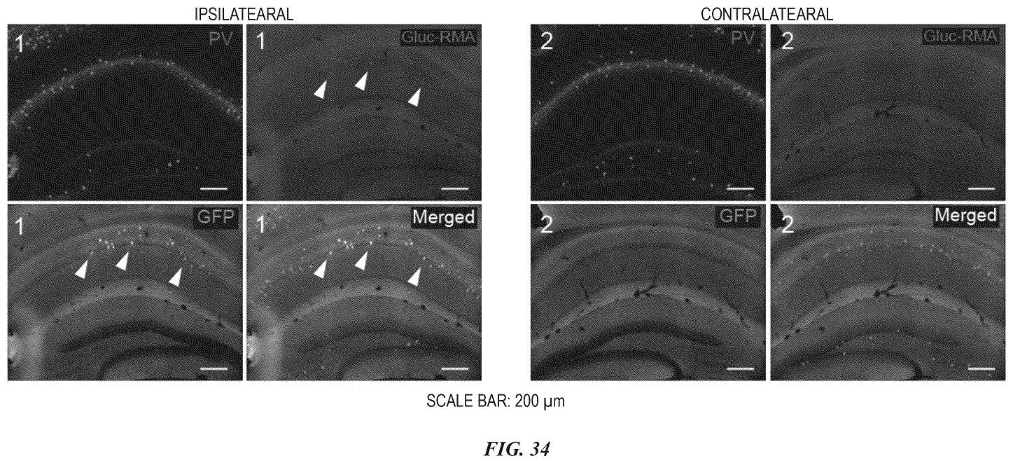

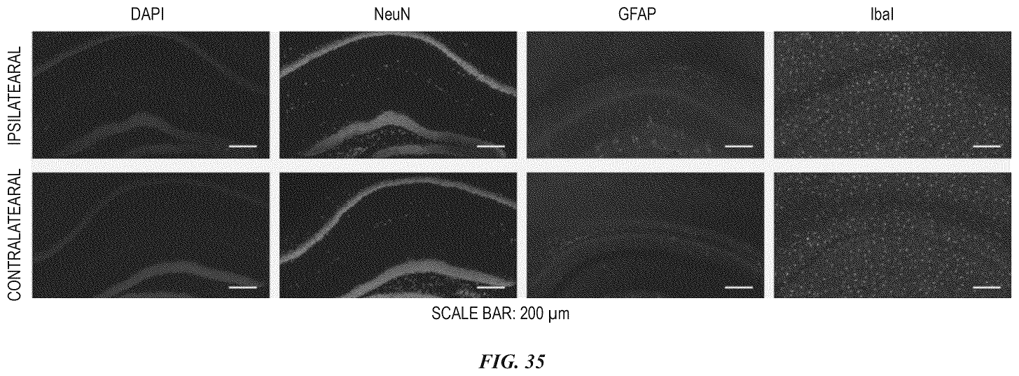

After 3 weeks post-delivery, the 1/100× dose resulted in a significant 38-fold increase in the plasma signals generated by approximately 125 (+92, n=4) transduced interneurons, without causing noticeable inflammation. This is illustrated with reference to FIGS. 33-36. In particular, FIG. 33 depicts plasma bioluminescence signal and a representative image of the stained brain slice showing the expression of Gluc-RMA and GFP among PV positive interneurons, where n=4 independent mice analyzed. FIG. 34 depicts enlarged views of CA1, corresponding to region 1 (ipsilateral) and region 2 (contralateral) of the rectangular boxes shown in FIG. 33. Triangle markers indicate the cells that express Gluc-RMA and GFP at the ipsilateral region. FIG. 35 depicts images stained to evaluate inflammation. In the depicted images, GFAP and IbaI were stained using separate brain slices.

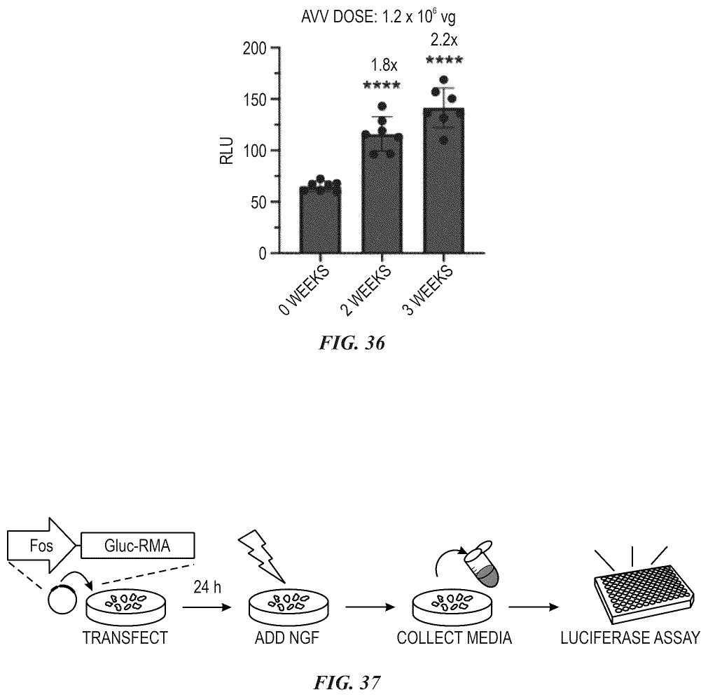

It was observed that reducing the dose by 10-fold (1/1000×) still produced detectable signals (2.2-fold increase at 3^{rd} week), indicating that Gluc-RMA exhibits high sensitivity that could be applicable to monitoring minute change in gene expression. This is depicted in FIG. 36, in which a bar graph depicts plasma bioluminescence signal obtained with reduced AAV dose based on n=7 independent mice analyzed. *P<0.05, **** P<0.0001, ns (not significant) in comparison with the signal at 0 weeks, using One-way ANOVA, Tukey's test. Data are shown as mean±SD.

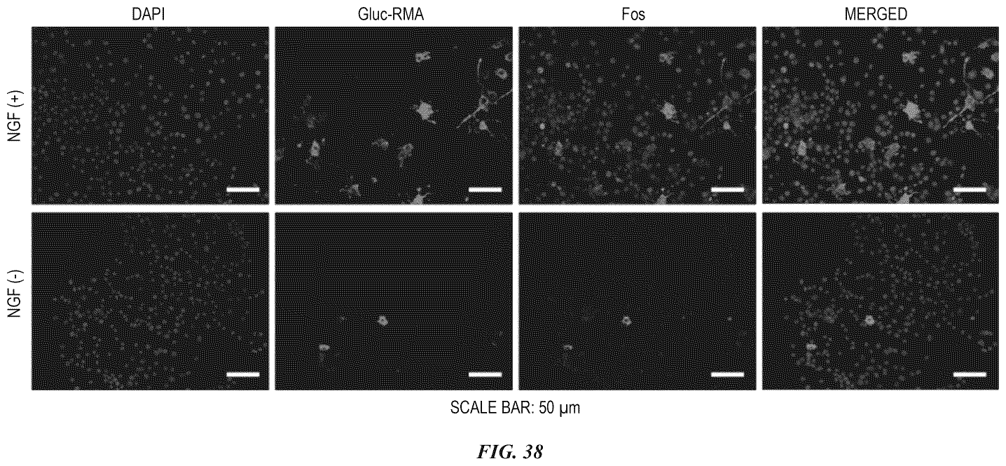

RMAs capture Fos gene expression activity—The ability of RMAs to track changes in the expression of the IEG Fos, which rapidly expresses upon cellular stimulus or neuronal activity, was also investigated. For this in vitro experiment, PC-12 was transfected with plasmid encoding Gluc-RMA controlled under the Fos promoter (Fos-Gluc-RMA). An example of this workflow is illustrated in FIG. 37, which depicts an experimental scheme in which PC-12 cells are transfected with plasmids encoding Gluc-RMA controlled under the Fos promoter, stimulated by NGF, and analyzed by luciferase assay for the secreted Gluc-RMA.

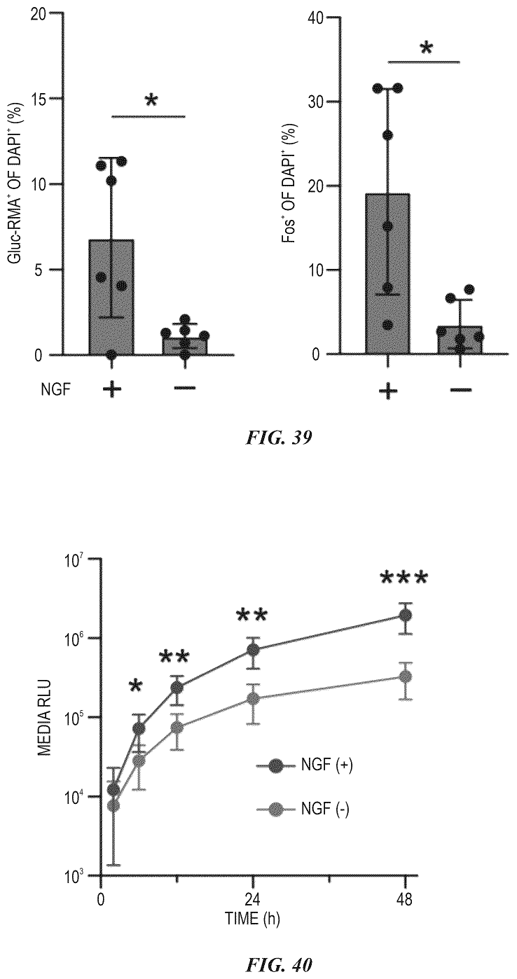

To induce Fos expression in transfected PC-12, the culture media was supplemented with nerve (e.g., neural) growth factor (NGF). Subsequently, luciferase assays were performed on the culture media from multiple time points, the results of which are illustrated in FIGS. 38 and 39. In particular, FIG. 38 depicts representative images of PC-12 stained 48 hr after the addition of media with or without NGF. FIG. 39 depicts, via graphs, the percentage of PC-12 cells that express Gluc-RMA (left) or Fos (right) for n=6 independent cultures analyzed. *P<0.05, **P<0.01, ***P<0.001, using unpaired two-tailed t-test. Data are shown as mean+SD.

Upon NGF induction, increased expression of Gluc-RMA and Fos was observed. Consistent with these results, the luminescence signal of the culture media rose significantly within 6 hr of exposure to NGF, suggesting that Gluc-RMA can generate a distinguishable signal output in response to changes in promoter activity. These results are illustrated in FIG. 40, where results for RLU measured from the culture media that contains the released Gluc-RMA are shown for n=6 independent cultures analyzed. Asterisks show the comparison between the RLU values with NGF (+) and without NGF (−) of the same time point. *P<0.05, **P<0.01, ***P<0.001, using unpaired two-tailed t-test. Data are shown as mean±SD.

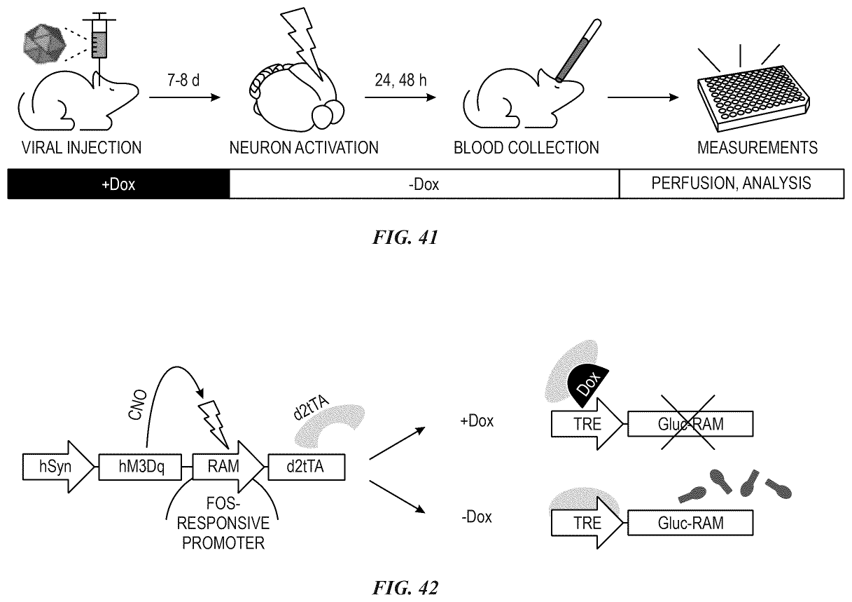

Noninvasive measurement of neuronal activity in specific brain regions—To determine whether RMAs could be used to detect neuronal activity in vivo, a double-conditional strategy was designed to link Gluc-RMA expression to neuronal activity in the brain. An example workflow of this strategy is illustrated in FIG. 41, which depicts an experimental scheme for detecting neuronal activity through blood tests with RMAs. In the depicted workflow, mice undergo injection of AAVs carrying RMA reporter genes, then induction of neuronal activation, and blood collection for measurement of the released RMA reporter.

To enable chemogenetic neuromodulation, a DREADD (designer receptor exclusively activated by designer drug) system was implemented. For the present purposes, the excitatory DREADD hM3Dq was chosen, which, when activated by intraperitoneally (i.p.)-administered clozapine-N-oxide (CNO), elicits robust neuronal firing and c-Fos accumulation. For the readout of plasma Gluc-RMA, a doxycycline (Dox)-dependent Tet-Off system called Robust Activity Marking (RAM) was incorporated to couple the RMA reporter gene to a synthetic Fos promoter and gain temporal control over its transcription, as shown in FIG. 42. In particular, FIG. 42 depicts a schematic of the RAM system for detecting neuronal activity. As shown in FIG. 42, upon CNO injection, activatory hM3Dq DREADD induces Fos expression. Subsequently, the RAM promoter drives the expression of d2tTA (tetracycline-controlled transactivator fused to a degradation domain) transactivator. If Dox is absent the d2tTA then binds to a tTA-responsive element (TRE) and induces the expression of Gluc-RMA. If present, Dox prevents d2tTA from binding to TRE and thus prevents the expression, allowing for temporally-gated recording of neuronal activity. Upon neuronal stimulation with CNO, both an active Fos promoter and the absence of Dox is needed to drive the expression of Gluc-RMA. To account for any variations in gene expression activities between mice, an internal control Cypridina luciferase (Cluc)-RMA constitutively expressed under the hSyn promoter was constructed, thus demonstrating multiplexed monitoring capability of RMAs.

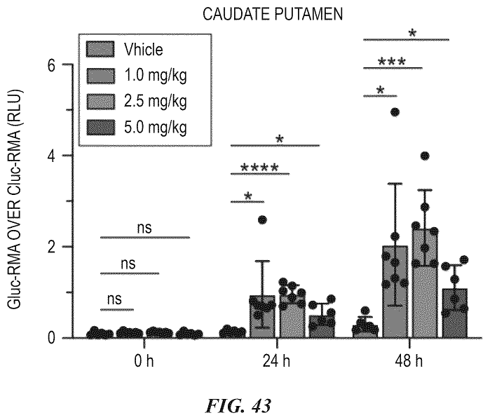

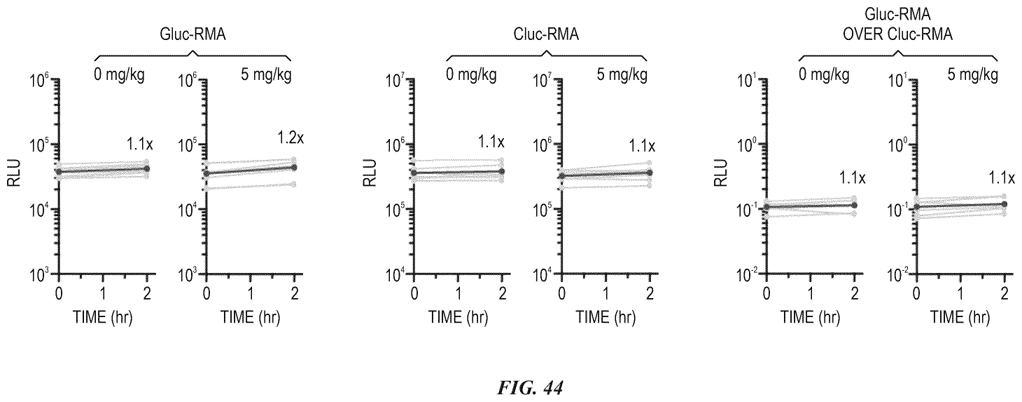

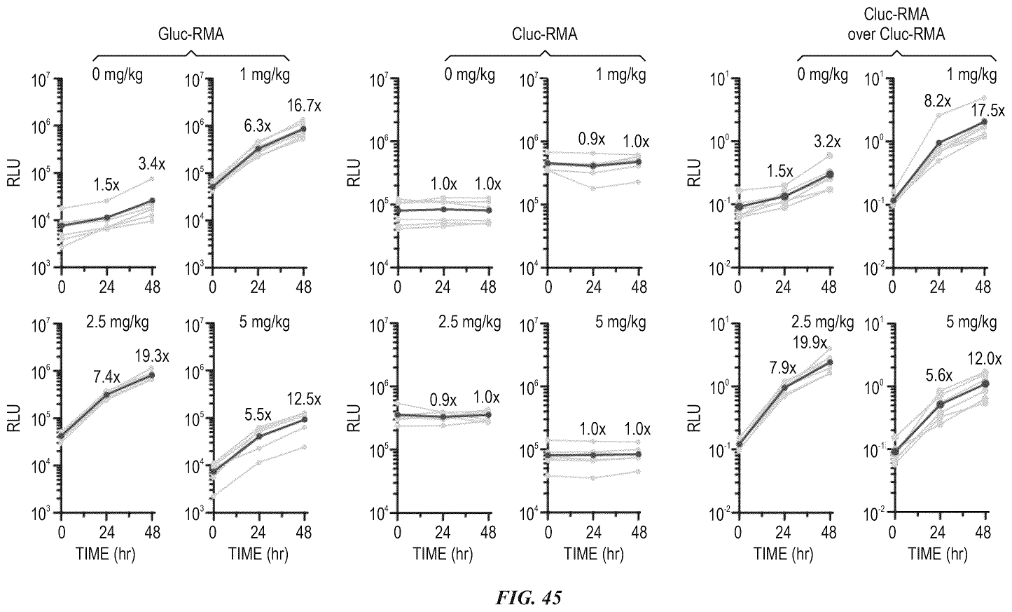

AAVs encoding the Fos-responsive, RAM-controlled Gluc-RMA-IRES-GFP were then prepared and delivered into the left CP of mice, along with Cluc-RMA and hM3Dq. The mice were fed a Dox chow diet, which was replaced with a Dox-free diet 48 hr prior to administering CNO for neuronal activation, as shown with respect to FIGS. 41 and 42. Results showed that mice injected with 5 mg/kg of CNO generated plasma Gluc-RMA signals that were 3.8-fold higher than the vehicle at 48 hr post-activation, whereas no signal difference was observed at 2 hr post-activation. These results are illustrated in FIGS. 43-45, where FIG. 43 depicts plasma bioluminescence signal of Gluc-RMA before (0 h) and after (24 h and 48 h) inducing chemogenetic activation at CP of the striatum with varying doses of CNO. Signals were normalized by dividing the RLU values obtained from Gluc-RMA over Cluc-RMA. n=6 to 7 independent mice analyzed, using two-way ANOVA, Sidak's test. *P<0.05, **P<0.01, ***P<0.001, ****P<0.0001. Data are shown as mean±SD. FIG. 44 illustrates average (dark line) and individual mice (light lines) plasma RLU values measured before and 2 h after the i.p. injection of either vehicle (0 mg kg⁻¹) or 5 mg kg⁻¹ of CNO. Normalized signal (right) was calculated by dividing the RLU values of Gluc-RMA by that of Cluc-RMA of the same mouse at the same time point for n=7 independent mice analyzed. FIG. 45 illustrates average plasma RLU values (dark line) calculated by those of individual mouse (light lines) measured after the i.p. injection of vehicle, 1, 2.5 or 5 mg kg⁻¹ of CNO for those chemogenetically activated at the left CP. Normalized signal (right) was calculated by dividing the RLU value of Gluc-RMA by that of Cluc-RMA of the same mouse at the same time point. n=5 to 7 independent mice analyzed.

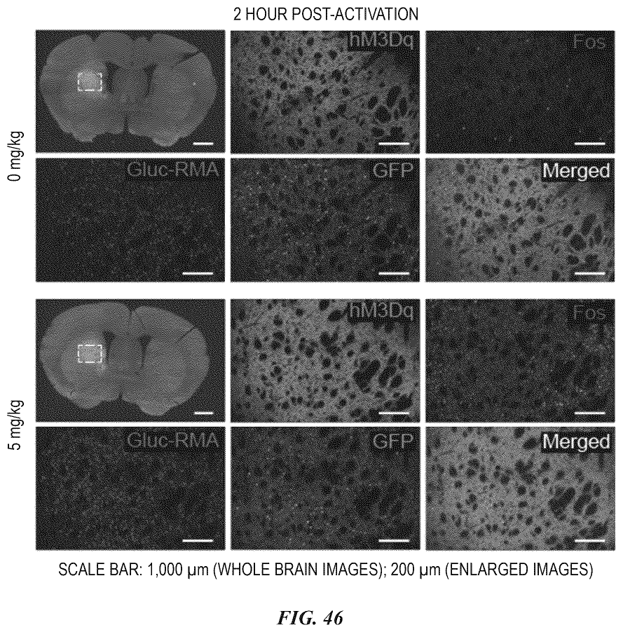

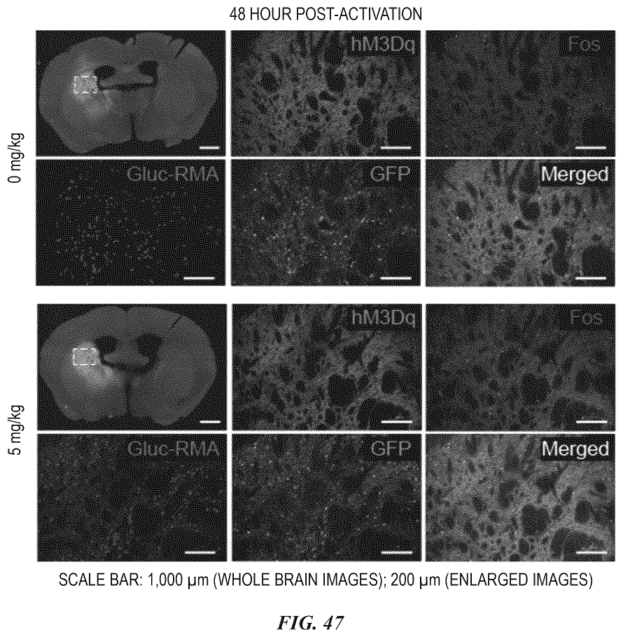

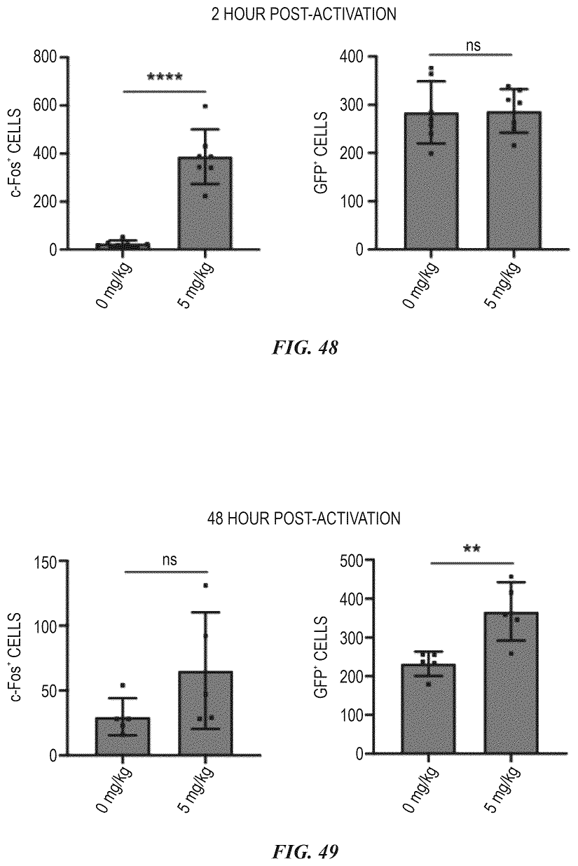

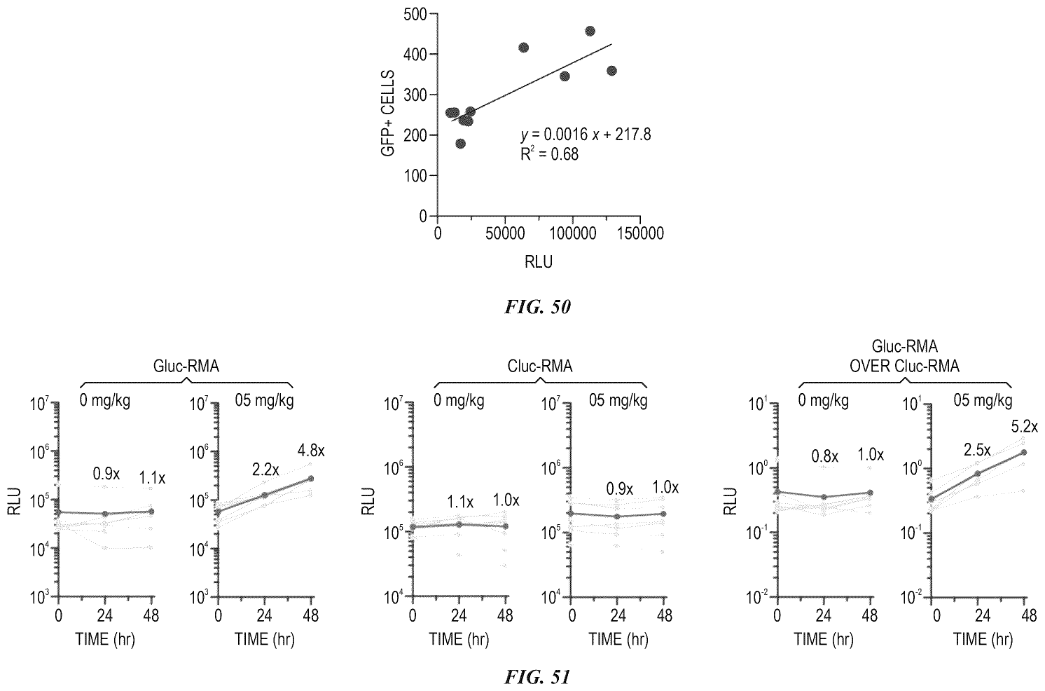

Similarly, histological analysis indicated that the number of cells expressing GFP for the CNO group revealed 1.6-fold higher than the vehicle group by 48 hr but not at 2 hr post-activation. This is illustrated in FIGS. 46-49. FIGS. 46 and 47 depict representative images of stained brain slices of mouse perfused after 2 hr (FIG. 46) and 48 hr (FIG. 47) post-injection of either vehicle or CNO. FIGS. 48 and 49 depict quantification of cells expressing c-Fos and GFP at 2 hr (FIG. 48) and 48 hr (FIG. 49) post-CNO injection analyzed using image groups represented in FIGS. 46 and 47, respectively. n=5 (for 48 hr) to 7 (for 2 hr) independent samples analyzed. **P<0.01, ****P<0.0001, ns (not significant), in comparison between the vehicle- and CNO-injected groups, using unpaired two-tailed t-test. Data are shown as mean±SD. However, the activation of c-Fos upon CNO administration showed significantly higher at 2 hr (15.6-fold) than at 48 hr (2.2-fold). These results suggest that the early c-Fos activation could lead to a delayed response in the detectable plasma Gluc-RMA signals and expression of GFP. The correlation between the number of cells expressing intracellular GFP and the corresponding Gluc-RMA signal obtained in each mouse at 48 hr post-activation was analyzed and a discernible correlation (r²=0.68) observed, as shown in FIG. 50 which depicts the correlation between the counted number of GFP positive cells in the image and the corresponding plasma bioluminescence signal of each mouse at 48 hr post-injection of either vehicle or CNO.

To gain a better understanding of the effectiveness of Gluc-RMAs in monitoring chemogenetic activation, lower CNO doses of 1 and 2 mg/kg were used in the same CP region. Interestingly, it was found that the increase in plasma Gluc-RMA signals was greater with these lower doses than with the 5 mg/kg dose, possibly due to the antipsychotic effect of the metabolite clozapine that may have caused sedation (FIG. 43 and FIG. 45. As expected, significant changes in the expression of Cluc-RMA across all doses were not detected (FIG. 45).

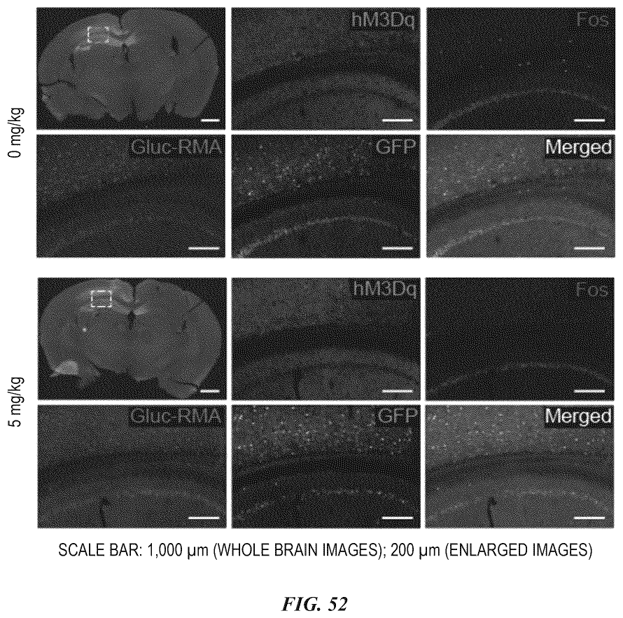

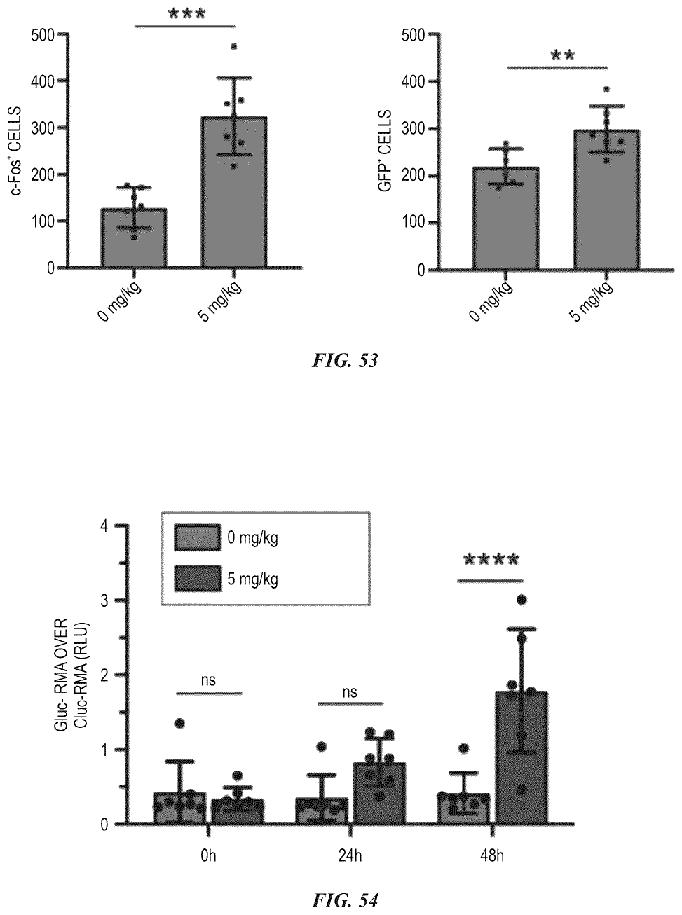

To examine applicability to a different brain region, testing was done in the CA1 of the hippocampus and a similar trend observed with a 4.6-fold increase in the plasma signal at 48 hr after CNO administration. Aspects of this study are illustrated in FIGS. 51-54. In particular, FIG. 51 depicts plasma RLU values presented analyzed in a similar format as shown with respect to FIG. 45, but with results obtained by conducting the study at the left hippocampus using vehicle and 5 mg kg⁻¹ of CNO. FIG. 52 depicts representative images of stained brain slices showing hippocampus of mouse perfused after 48 hr post-CNO injection. FIG. 53 depicts graphs illustrating quantification of cells expressing c-Fos and GFP analyzed using image groups represented in FIG. 52, with n=7 independent samples analyzed, using unpaired two-tailed t-test, in comparison between the vehicle- and CNO-injected groups. FIG. 54 illustrates the plasma bioluminescence signal of Gluc-RMA normalized to Cluc-RMA upon chemogenetic activation in the left hippocampus, based on. n=7 independent mice analyzed, using two-way ANOVA, Sidak's test. **P<0.01, *** P<0.001, ****P<0.0001, ns (not significant). Data are shown as mean+SD. Collectively, the findings demonstrate the multiplexed ratiometric measurements of RMAs as well as their ability to discriminately report on in vivo neuronal activity of specific brain regions.

RMA enhances in vivo bioluminescence imaging (BLI)—Whether Gluc-RMA could be used to improve BLI was also studied. In vivo imaging system (IVIS) allows for noninvasive imaging of cells or tissues of interest using optical sensors. Due to the poor penetration of light in vivo, researchers commonly rely on albino or nude animals or high concentrations of reporters and typically limit their studies to small animal species. Additionally, the choice of luminophore is limited by the need of those molecules to cross the BBB. Because Gluc-RMA can be released from the brain and has a long t_{1/2} in the blood, a study was conducted as to whether Gluc-RMA could be used with BLI to facilitate the measurement of gene expression levels within the brain areas transduced with Gluc-RMA.

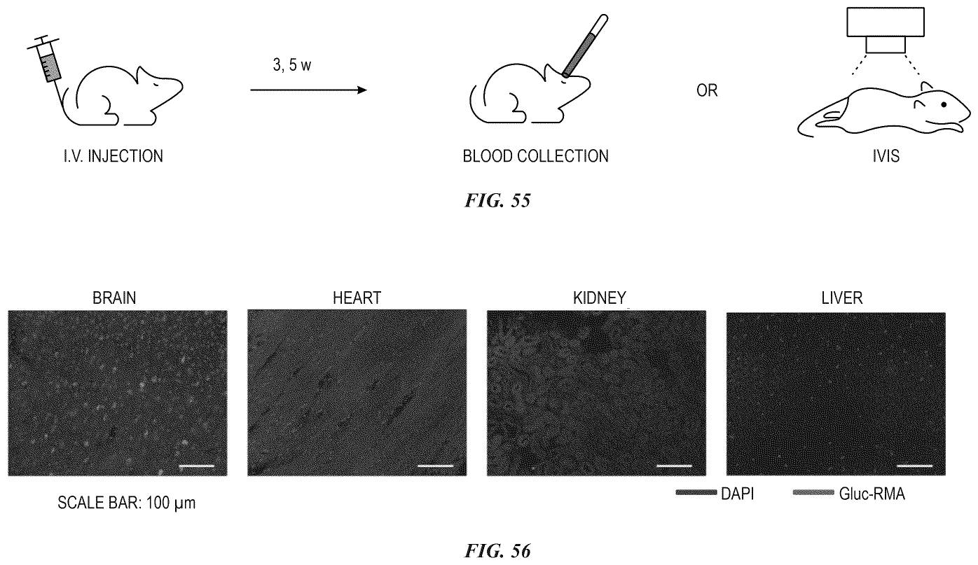

Gluc or Gluc-RMA was expressed in the whole brain by intravenously (i.v.) injecting BBB-permeable PHP.eB AAV and then the mice were imaged or their plasma analyzed using IVIS or luciferase assay, respectively. By way of example of this workflow, FIG. 55 depicts a schematic of delivery of the BBB-permeable PHP.eB virus encoding Gluc-RMA to the brain through intravenous (i.v.) injection and subsequent collection of the blood or BLI for measurement of the brain gene expression. FIG. 56 depicts Gluc-RMA expression in the brain, heart, kidney, and liver at 5 weeks post-injection of 5.0×10⁹ vg/g PHP.eB. The stained images show the expression occurs in the brain but not in other organs.

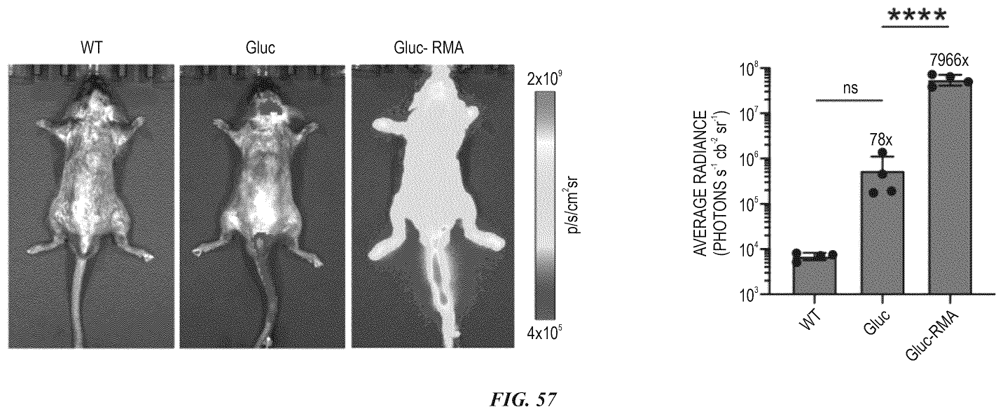

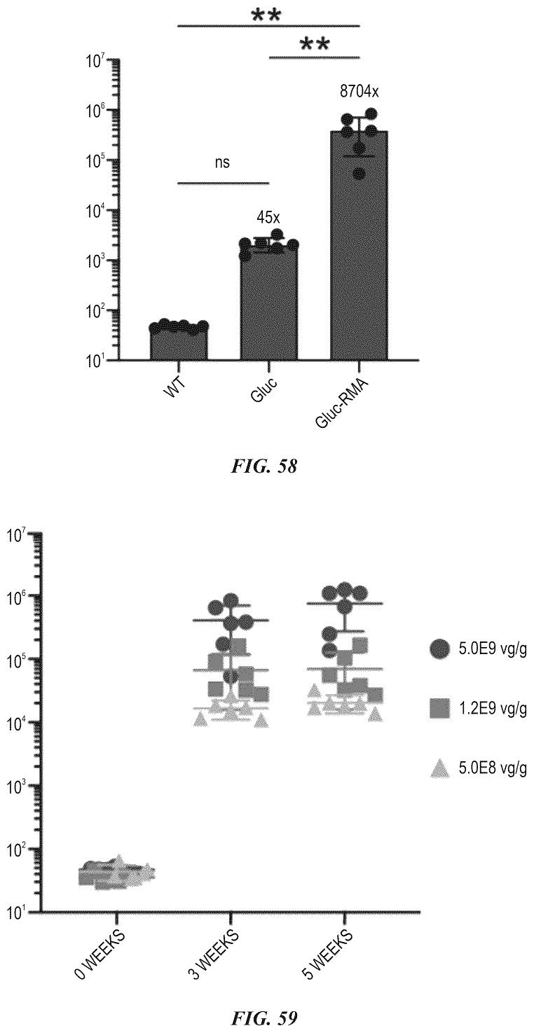

Gluc-RMA improved the IVIS photon emission by a factor of 102 over Gluc, which showed detectable but not significant signal against the wild-type (WT). This is illustrated in FIG. 57, which illustrates bioluminescence imaging of mice taken after 3 weeks post-PHP.eB delivery at the dose of 5.0×10⁹ vg g⁻¹ mouse. 12 μmol kg⁻¹ of native coelenterazine was injected through the tail-vein and immediately imaged under the IVIS. Average radiance of the upper body was quantified using the Living Image software and n=4 independent mice were analyzed where **P<0.01, ****P<0.0001, ns (not significant), using one-way ANOVA, Tukey's multiple comparison test. A similar trend was observed in the plasma assays showing 193-fold improvement over Gluc. This is illustrated in FIG. 58, which depicts plasma RLU measured at 3-week post-PHP.eB delivery of the same viral dose, and where **P<0.01, ****P<0.0001, ns (not significant), using one-way ANOVA, Tukey's multiple comparison test and for n=6 independent mice analyzed. Data are shown as mean±SD.

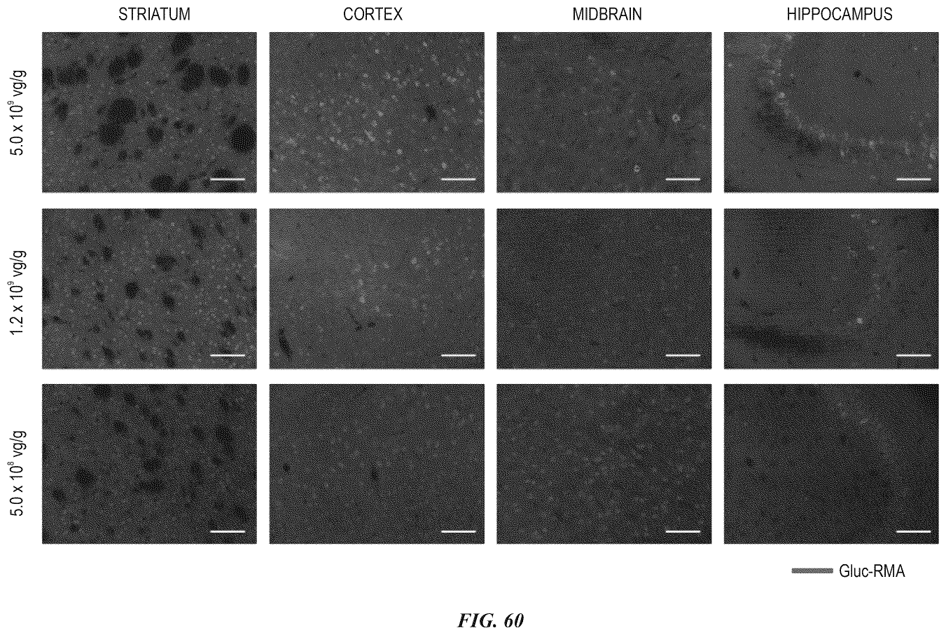

Furthermore, the study confirmed that Gluc-RMA signals are correlated to gene expression levels through an AAV dose response analysis, aspects of which are shown in FIGS. 59 and 60. In particular, FIG. 59 illustrates the PHP.eB dose response to the plasma bioluminescence signal for n=6 independent mice analyzed. Data are shown as mean±SD. WT: wild type. FIG. 60 depicts images of Gluc-RMA expression in the striatum, cortex, midbrain, and hippocampus in the brain of mice administered with different PHP.eB doses. Taken together, these data suggest that Gluc-RMA improves the imaging performance of BLI by substantially enhancing its signal intensity.

Discussion—As described in the results above, RMAs appear suitable as a new class of reporters to noninvasively measure gene expression in the brain. In particular, the presently described example of RMAs is suitable for high-sensitivity detection. As discussed herein, to achieve this, an endogenous pathway was repurposed that allowed transport of RMAs from the brain to the blood. While FcRn is expressed in various tissues, the inclusion of the Fc region in RMA, when used as a brain reporter, facilitates a dual function of enabling it to cross the BBB and prolonging its lifetime in the blood. RMAs accumulate in the blood over time and, owing to their long half-life, avoid the rapid clearance or low concentrations commonly encountered by natural brain-derived biomarkers. RMAs are thus versatile gene expression reporters that can be expressed under any suitable promoter of interest to monitor long-term changes in gene expression or efficiency of gene delivery to the brain in individual animals. Such long-term gene expression changes can be observed, for example, in tracking the dynamics of neuronal subtypes, brain disorder pathogenesis, aging, or transgene expression following gene therapy administration.

Since the brain is completely vascularized, RMAs can be used in any region of the brain, regardless of whether that region is deep or cortical. Thus, RMAs avoid the obstacles faced by many other methodologies that are limited by the depth of penetration, tissue scattering, or skull absorption of the penetrant waves used to image reporters. Signal levels between ˜20,000-40,000-fold over the baseline have been demonstrated in three commonly studied brain regions after a single intracranial injection of AAVs carrying RMAs. RMAs could be of utility in large animal models where tissue scattering or skull absorption preclude the use of optical systems, such as intravital BLI. Because of its physical accessibility, blood has been commonly used for diagnosing various medical conditions, including cancer and neurodegenerative diseases. Therefore, if effective in humans, blood assays for RMA could be clinically adopted to provide patients with a more convenient option over noninvasive techniques that image directly on the brain. Lastly, RMAs democratize access to noninvasive measurement of gene expression in the brain, opening this technique for use, for example, in high-throughput screening scenarios. Readout of RMAs does not require complicated scanners, such as MRI, because it relies on serum chemistry that is accessible to many research laboratories.

The expression of RMAs is influenced by the cell type and spatial specificity, which, in turn, depends on the method of delivery. Although intracranial injection is invasive, it is commonly used in research and has been accepted in numerous clinical trials for region-specific delivery. However, due to its limitations in the number of injections, it may not be ideal for large area delivery. To address this challenge, the present studies have demonstrated that the evolved PHP.eB AAV with the neuron-specific promoter can facilitate whole-brain delivery in a tissue- and cell-type specific manner, as demonstrated by FIGS. 56 and 60. Recent studies have shown that noninvasive viral delivery can achieve millimeter precision in both local and large brain areas using Focused Ultrasound-BBB Opening (FUS-BBBO). This method temporarily opens the BBB by creating stable cavitation of microbubbles within the microvessels. FUS-BBBO offers a promising alternative for noninvasive, spatially and molecular-specific delivery of RMAs. In addition, the presently described example of a Gluc-RMA design, with a length of approximately 1.2 kB, provides ample space to accommodate short therapeutic genes, such as the gene editing enzymes of small CRISPR-based Cas effectors (400 to 800 amino acids), within the 4.7 KB AAV packaging capacity. Longer payloads, however, may be delivered using a larger cargo or complementary AAV to split the expression cassette into two vectors, allowing for self-annealing to form a full-length DNA after delivery. Based upon the presently disclosed subject matter and studies, it is believed that the ongoing efforts to develop versatile delivery platforms will enable targeted delivery of RMAs with high specificity for a wider range of use cases in the brain.

Multiplexed RMA readout using Gluc-RMA and Cluc-RMA, which react with different substrates to emit different bioluminescence signals have been demonstrated. Gluc or Cluc were selected for their ability to be secreted and conveniently assayed, but one could instead construct a compound of secreted library proteins fused to Fc to implement a highly multiplexed RMA system. In combination with highly multiplexed protein detection methods, RMAs have the potential to achieve higher multiplexity than currently available noninvasive methodologies because their readout relies on biochemical methods. If such multiplexed monitoring is implemented, RMAs could be used to independently monitor large numbers of cells or genes. This ability for high multiplexity could confer an advantage on using RMAs over other techniques that rely on the limited number of reporter variants or fluorescent channels available.

The ability to monitor gene expression activities of individual or a subset of neuronal populations with high sensitivity will help with discriminating different functional networks in the deep brain. It was determined that 815 neurons were sufficient to produce 43-fold higher RMA signals compared with the baseline, as shown in FIG. 23. It may also be noted that the experiment with the 1,000-fold reduction in AAV dose could have resulted in a lower MOI (multiplicity of infection) relative to the described high dose experiments, suggesting that sensitivity could be greater in some cases, e.g., transduction of sparse cell populations with high doses of the AAV. The plasma signals (around 2×10³ RLU) were one order of magnitude lower than the expected RLU values (815 neurons×26.0≈2×10⁴ RLU) based on its linear relationship to the number of transduced neurons, as shown in FIG. 24, indicating that gene expression levels per neuron were comparatively low with the reduced AAV dose. These results support the idea that Gluc-RMA could report on even fewer neurons if small numbers of cells can be transduced with a high concentration of the AAV.

The difference in the levels of plasma Gluc-RMA after chemogenetic activation began to appear after 24 hr, consistent with the time taken for fluorescent reporters to express in the brain cells using the RAM system. In contrast, an observable rise in c-Fos was evident after only 2 hr in histology (˜15-fold), and this increase was similar to the rise in bioluminescence signals by 48 hr (˜4 to 5-fold). These data suggest that the expression of the reporter protein, including transcription and translation processes, accounts for the major time delay in detecting the output signals. In addition, given their long half-life, the current RMAs are less applicable to transient neuronal activities or studies that require high temporal resolution and are more useful for measuring persistent changes in the timescale of days. To increase the temporal resolution, the expression of RMAs may be accelerated and their lifespan in the blood shortened without compromising the Fc-dependent release from the brain. The application of emerging single-molecule protein detection methods could allow RMAs to both achieve faster readout kinetics and maintain the high sensitivity observed in this study.

With the RMA platform, the use of blood as an alternative route for measuring gene expression in the intact brain has been demonstrated. RMAs are genetically-encodable reporters that exhibit high sensitivity, repeatability, and multiplexity, making them well-suited for numerous neuroscience applications, such as monitoring differential gene expression activities among cell type-, circuit-, or spatially-specific brain cells, observing long-term changes in different neuronal subtypes, or monitoring changes in neuronal activity. It is anticipated that by opening a new noninvasive pathway into the brain, RMAs will prove to be an invaluable and promising tool for brain gene expression studies.

Methods—Animal subjects-Wild—type C57BL/6J (Strain #000664) and transgenic TH-Cre (Strain #008601) and PV-Cre (Strain #017320) male and female mice at 8-10 weeks old were purchased from the Jackson Laboratory. Animals were housed with a 12 h light-dark cycle and were provided with food and water ad libitum. All animal experiments were performed under the protocol approved by the Institutional Animal Care and Use Committee of Rice University.

Methods-Plasmid construction—To construct AAV-hSyn-RMA (Gluc-Fc), the vector AAV-hSyn-GlucM23-iChloC-EYFP (Addgene #114102) was digested with KpnI and EcoRV (New England Biolabs) to isolate the backbone containing the hSyn promoter. GlucM23, a Gluc variant, was amplified by PCR from the same vector and its DNA was extracted using the Monarch DNA Gel Extraction Kit (New England Biolabs). DNA segments for Fc regions, including the human IgG1 Fc (Addgene #145165), and mouse IgG1 Fc (Addgene #28216) and IgG2a Fc (Addgene #114492), were amplified and extracted similarly. Gluc alone or Gluc with Fc was inserted into the digested backbone through Gibson Assembly. To make mFc from the human IgG1 Fc, the relevant mutations were introduced using site-directed mutagenesis. For RMA controls that lack the signal peptides, the first 17 amino acids of the Gluc sequence were skipped during the amplification. To co-express RMA and GFP, IRES-GFP sequence from the bicistronic vector (Addgene #105533) was amplified and inserted downstream of the RMA coding region to construct AAV-hSyn-RMA-IRES-GFP. To construct AAV-GFAP-RMA, the GFAP promoter was extracted from the vector AAV-GFAP-mKate2.5f (Addgene #99129) and inserted together with the RMA segment into the backbone obtained from AAV-hSyn-RMA-IRES-GFP digested with BamHI and EcoRV. Plasmid for RMA controlled under the Fos promoter AAV-Fos-RMA was constructed by extracting the Fos promoter from the plasmid Fos-tTA (Addgene #34856) and replacing the hSyn with the Fos promoter from the presently described AAV-hSyn-RMA. For plasmids that encode Cluc-RMA (Cluc-Fc), the Cluc DNA was obtained from pClucIPZ (Addgene #53222) and used instead of Gluc for assembly. To construct AAV-hSyn-DIO-RMA-IRES-GFP, the DIO sequence that contains lox2272 and loxP was extracted from AAV-hSyn-DIO-hM3D (Gq)-mCherry (Addgene #44361) and assembled with the reversed RMA-IRES-GFP sequence into the previously digested backbone from AAV-hSyn-RMA-IRES-GFP.

To make pET-T7-RMA-His for purifying RMA proteins, the RMA sequence was amplified from the presently described AAV-hSyn-RMA. His tag was attached to the C-terminus of RMA using reverse primers containing the overhang that encodes six His residues. To construct non-FcRn-binding RMA, mutations I203A+H260A+H385A were introduced using site-directed mutagenesis into the vector pET-T7-RMA. The pET28a vector was provided by the Tabor Lab at Rice University. The amplified DNA was then inserted into the pET28a backbone using Gibson Assembly.

To construct AAV-hSyn-hM3Dq-RAM-d2tTA, AAV-hSyn-hM3Dq-mCherry (Addgene #50474) was digested with SalI and PmlI to obtain the backbone that contains the hSyn promoter. hM3Dq was amplified separately to add an HA tag to its N-terminus. RAM-d2tTA was amplified and extracted from AAV-RAM-d2tTA-TRE-MCS (Addgene #63931). Two inserts HA-hM3Dq and RAM-d2tTA were then assembled into the backbone. For AAV-TRE-RMA-IRES-GFP, AAV-RAM-d2tTA-TRE-MCS was digested with NheI and KpnI and the segment RMA-IRES-GFP was used as an insert for Gibson Assembly.

Methods-PC-12 culture for luciferase assay-PC-12 (ATCC) was cultured in RPMI 1640 medium (Corning) supplemented with heat-inactivated 10% horse serum (Life Technologies) and 5% fetal bovine serum (FBS) (Corning). Cells were incubated in humidified air with 5% CO2 at 37° C. and split every 2 d with a subcultivation ratio of 1:2 or 1:3.

For in vitro luciferase assay, PC-12 was seeded at 200,000 cells per well in a 12-well plate. After 16-20 h, 1,500 ng of plasmids encoding hSyn-RMA and 3.0 μl of lipofectamine 2000 (Life Technologies) were used to transfect PC-12 following the manufacturer's protocol. Then, 25 μl of the culture media were collected at different time points and stored in −20° C. until use. For Gluc substrate, 0.5 mM native coelenterazine (CTZ) stock (Nanolight Technology) was dissolved in luciferase assay buffer (10 mM Tris, 1 mM EDTA, 1.2 M NaCl, pH=8.0) containing 66% DMSO and stored at −80° C. Before measuring bioluminescence, the CTZ stock was diluted to 20 M in luciferase assay buffer and kept in dark at room temperature for 1 h. Media samples were thawed in ice and transferred to black 96-well plate (Corning). Infinite M Plex microplate reader (Tecan) was used to inject 50 μl of the assay buffer containing CTZ and measure the photon emission integrated over 30 s. Values were averaged to obtain the light unit per second.

Methods—Astrocyte culture for luciferase assay—Dissociated cells from E-18 Sprague Dawley rat cortex (TransnetYX Tissue) were cultured under the NbASTRO glial culture medium (TransnetYX Tissue) in T75 flask coated with Poly-D-Lysine (PDL) (Thermo Fisher Scientific) and incubated in humidified air with 5% CO₂ at 37° C. The media was refreshed every 3 d and neurons were starved over a period of 1 w to isolate the astrocytes. For luciferase assay, astrocytes were seeded at 16,000 cells per well in a PDL-coated 12-well plate. After 16-20 h, 500 ng of plasmids encoding GFAP-RMA and Lipofectamine LTX with Plus Reagent (Thermo Fisher Scientific) were used to transfect astrocytes following the manufacturer's protocol. Cells were replaced with fresh medium 6 h post-transfection. As in the PC-12 experiment, 25 μl of the culture media were collected at different time points to conduct luciferase assay.

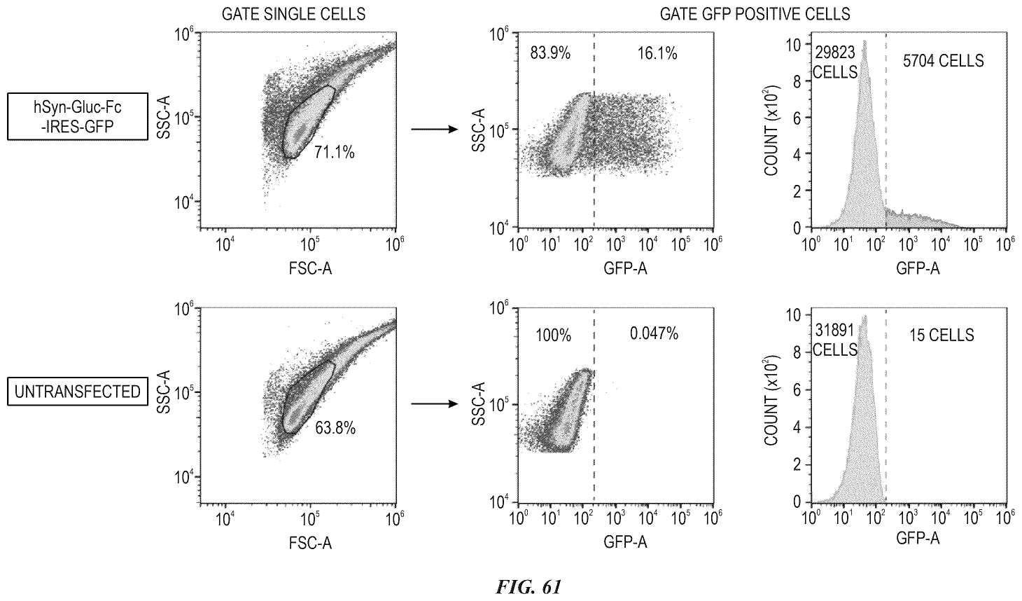

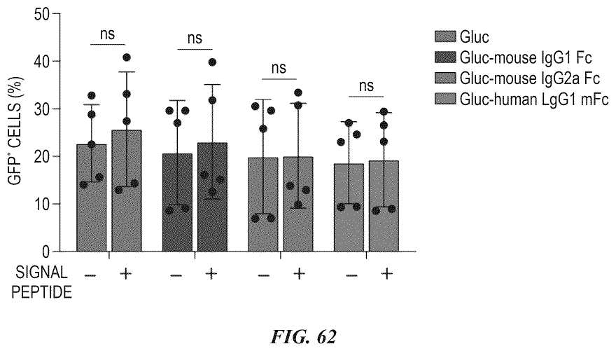

Methods—Measurement of RMA release per cell—PC-12 was transfected using the plasmid hSyn-RMA-IRES-GFP. After 72 h, the total cell number in each well was estimated using 10 μl of the culture media mixed with Trypan Blue and by counting the viable cells under the hemocytometer (INCYTO). The remaining cells and media were then centrifuged and processed separately. Cells were analyzed by flow cytometer (Sony SA3800) with at least 50,000 events to obtain the percentage of GFP positive cells. Non-transfected PC-12 was used to gate the positive cells. Aspects of this are illustrated in FIGS. 61 and 62. FIG. 61 depicts the gating strategy for estimating the percentage of GFP positive cells. PC-12 cells were transfected with bicistronic vector encoding RMA and GFP. After 72 h post-delivery, spectral cell analyzer was used to sequentially gate single cells and then the GFP positive cells against the untransfected control. The data illustrated in FIG. 61 represent a sample of PC-12 cells expressing Gluc-mouse IgG1 Fc and GFP versus the untransfected control. FIG. 62 illustrates the percentage of PC-12 cells expressing GFP analyzed by FACS. ns (not significant) in comparison with the percent GFP positive cells of the respective RMA, using two-way ANOVA Sidak's test. Data are shown as mean±SD. The number of transfected cells was calculated by multiplying the total cell number to the GFP⁺ percentage. For processing the media, luciferase assay was conducted to obtain their bioluminescence signals and a standard curve was generated using the fresh culture media spiked with purified RMA proteins at different concentrations. The total secreted RMAs was calculated by multiplying the concentration obtained from the standard curve to the total media volume. Finally, the number of RMAs released per cell was calculated by dividing the secreted RMAs over the number of transfected cells.

Methods—Protein purification—Shuffle T7 Express chemically competent E. coli cells (New England Biolabs) were transformed using the plasmid pET-T7-RMA-His. The next day, a single colony was transferred into 3 ml of the LB medium as a starter culture to grow overnight at 30° C. under 250 RPM in a shaker. The culture was then transferred into 1 L Terrific broth and grown in the same condition until the optical density at 600 nm reached 0.5. The culture flask was cooled on ice for 30 min, supplemented with 100 μM IPTG, and induced for 20 h at 16° C. under 180 RPM. Cells were harvested by centrifugation at 4,000 g for 20 min, resuspended in lysis buffer (300 mM NaCl, 50 mM NaH₂PO₄, 10 mM imidazole, 10% glycerol, pH 8.0) supplemented with ProBlock Gold protease inhibitor (Gold Biotechnology), and lysed by the sonicator (VCX 130, Sonics and Materials). Lysates were centrifuged at 12,000 g for 30 min at 4° C. The resulting supernatant was subject to binding with Ni-NTA agarose resin (Qiagen) for 30 min at 4° C. with gentle rotation and loaded into the glass chromatography columns (Bio-Rad) for wash and elution through gravity flow. Protein-bound resin was washed sequentially using lysis buffer with incremental increase of imidazole concentrations. Bradford protein assay (Thermo Fisher Scientific) was performed throughout the washing procedure to check for the presence of non-specific proteins. RMA proteins were eluted using lysis buffer containing 500 mM imidazole and buffer exchanged into PBS using the Amicon centrifugal filter unit with 10 kDa cutoff (MilliporeSigma). The final protein in PBS was analyzed by the SDS-PAGE. BCA protein assay (Thermo Fisher Scientific) was used to determine protein concentration.

Methods—Adeno-associated virus production—PHP.eB AAV virus was packaged by adopting a previously published protocol with slight modifications. In brief, HEK293T cells (ATCC) were transfected with the transfer plasmid PHP.eB iCap (Addgene #103005), and pHelper plasmids. After 24 hr, cells were exchanged with a fresh DMEM (Corning) supplemented with 5% FBS and non-essential amino acids (Life Technologies). At 4 d post-transfection, cells were harvested, and media were mixed with 1/5 volume of PEG solution (40% PEG 8,000, 2.5 M NaCl) to precipitate AAV at 4° C. for 2 hr. Cells were resuspended in PBS and lysed by the freeze-thaw method. The precipitated AAV was pelleted by centrifugation, resuspended in PBS, and combined with the lysed cells. The combined lysate was added with 50 U ml⁻¹ of Benzonase (Sigma-Aldrich) and incubated at 37° C. for 45 min before being stored at −20° C. for no more than one week.

AAV purification was carried out by the iodixanol gradient ultracentrifugation. Quick-seal tube (Beckman Coulter) was loaded with the iodixanol gradients (Sigma-Aldrich), including 60%, 40%, 25%, and 15%. The frozen lysate was thawed and centrifuged at 2,000 g for 10 min. The resulting clarified lysate was transferred on top of the iodixanol layers drop-by-drop. The tube was sealed and centrifuged at 58,400 RPM for 2.5 h using the 70 Ti fixed-angle rotor of an ultracentrifuge (Beckman Coulter). AAV was collected by extracting the 40%-60% iodixanol interface and washed using the Amicon centrifugal filter unit with 100 kDa cutoff (MilliporeSigma). The final AAV was filtered by passing through the 0.22 μm PES membrane. Viral titers were determined using the qPCR method.

Methods-Stereotaxic injection-Protein or AAV was injected into the mice brains using a microliter syringe equipped with a 34-gauge beveled needle (Hamilton) installed to a motorized pump (World Precision Instruments) using a stereotaxic frame (Kopf). To inject RMA protein, 20 μM of RMA was injected bilaterally to CP (AP+0.25 mm, ML±2.0 mm, DV-3.2 mm, 1 μl per hemisphere) infused at a rate of 200 nl min⁻¹ and the needle was kept in place for 5 min before taking it out from the injection site. For AAV injections, PHP.eB serotype was used for all experiments. To deliver AAV encoding hSyn-RMA-IRES-GFP, 2.4×10⁹ vg in 200 nl was injected per site at 600 nl min⁻¹ to the following coordinates: CP in the striatum (AP +0.25 mm, ML +2.0 mm, DV −3.2 mm), CA1 in the hippocampus (AP −1.94 mm, ML +1.0 mm, DV −1.3 mm), and substantia nigra in the midbrain (AP −3.28 mm, ML +1.5 mm, DV −4.3 mm). For conducting the 1/100^{th} and 1/1000^{th} dilution of the initial dose (2.4×10⁷ vg and 2.4×10⁶ vg, respectively), 50 nl was injected at 150 nl min⁻¹ into the same CP coordinate. For testing the TH-Cre and PV-Cre mice, AAV encoding hSyn-DIO-RMA-IRES-GFP was injected into the left VTA (AP −2.9 mm, ML +0.8 mm, DV −4.55 mm) and CA1 (AP −1.94 mm, ML +1.0 mm, DV −1.3 mm), respectively, at doses of 1.2×10⁹ vg for TH-Cre and 1.2×10⁷ vg or 1.2×10⁶ vg for PV-Cre.

For chemogenetic neuromodulation experiments, mice were placed on 40 mg kg⁻¹ of Dox chow (Bio-Serv) 24 h prior to surgery. AAV doses used are as follows: 2.0×10⁹ vg (hSyn-hM3Dq-RAM-d2tTA), 2.0×10⁹ vg (TRE-Gluc-RMA-IRES-GFP), and 1.1 ×10⁹ vg (hSyn-Cluc-RMA). For each surgery, AAV cocktail was prepared in 450 nl and injected over 1 min. The needle was kept at the injection site for 10 min owing to the relatively high volume of the cocktail. Dox chow was removed 48 h prior to inducing chemogenetic activation.

Methods—Blood collection for luciferase assay—Mice were anesthetized in 1.5%-2% isoflurane in air or O₂. Following, 1-2 drops of 0.5% ophthalmic proparacaine were applied topically to the cornea of an eye. Heparin-coated microhematocrit capillary tube (Fisher Scientific) was placed into the medial canthus of the eye and the retro-orbital plexus was punctured to withdraw 50-100 μl of blood. The collected blood was centrifuged at 1,500 g for 5 min to isolate plasma and stored at −20° C. until use. To conduct luciferase assay, 5 μl of plasma was mixed with 45 μl of PBS+0.001% Tween-20 in a black 96-well plate. Using the microplate reader, bioluminescence of Gluc-RMA or Cluc-RMA was measured by injecting 50 μl of 20 μM CTZ or 1.0 μM vargulin (Nanolight Technology), respectively, dissolved in the luciferase assay buffer into the plasma sample.

Methods—Serum half-life measurement—Mice were administered i.v. with 2 mg kg⁻¹ of purified RMA proteins. Retro-orbital blood collections were performed at specific time points. Concentrations of RMAs in the collected blood were determined using the standard curves generated by the luciferase assays conducted on the normal mouse serum (Sigma-Aldrich) added with RMAs at known concentrations. To determine the pharmacokinetic parameters, AUC^{inf} (area under the curve to infinity) was calculated using the log-linear trapezoid method³⁷. Half-life (t_{1/2}) of the distribution (α) or elimination (β) phase in the two-phase clearance model was calculated by applying the log-linear regression to the concentration data. The concentrations of the first three and the subsequent time points were used for determining the α- and β-phase t_{1/2}, respectively. Clearance was calculated by dividing the dose by AUC_{inf}.

Methods—Drug administration—Water-soluble CNO (Hello Bio #HB6149) were dissolved in saline (Hospira) at 1 mg ml⁻¹ and stored at −20° C. until use. To induce chemogenetic activation of mice expressing hM3Dq, CNO was injected i.p. at 1, 2.5, or 5 mg kg⁻¹ dose. For the vehicle (0 mg kg⁻¹) groups, saline was injected i.p. at the volume dose of 5 ml kg⁻¹.

Methods—Histological imaging and analysis—Mice brains were extracted and postfixed in 10% neutral buffered formalin (Sigma-Aldrich) overnight at 4° C. Coronal sections were cut at a thickness of 50 μm using a vibratome (Leica) and stored at 4° C. in PBS. Sections were stained as follows: 1) block for 2 h at room temperature with blocking buffer (0.2% Triton X-100 and 10% normal donkey serum in PBS); 2) incubate with primary antibody overnight at 4° C.; 3) wash in PBS for 15 min 3 times; and 4) incubate with secondary antibody for 4 h at room temperature. After last washes in PBS, sections were mounted on glass slides using the mounting medium (Vector Laboratories) with or without DAPI and cured overnight in dark at room temperature. Antibodies and dilutions used are as follows: rabbit anti-Gluc (1:1,500, Nanolight Technology), mouse IgG2a anti-Fos (1:500, Santa Cruz), mouse IgG2b anti-NeuN (1:1,500, Novus Biologicals), chicken IgY anti-IbaI (1:500, Synaptic Systems), mouse IgG2b anti-GFAP Alexa Fluor 647 (1:200, Santa Cruz), mouse IgG2b anti-IL-6 (1:200, Santa Cruz), chicken IgY anti-TH (1:1,000, Aves Labs), guinea pig anti-PV (1:500, Synaptic Systems), mouse IgG1 anti-HA-594 (1:500, Life Technologies), and Alexa 350, 488, 594, 647 secondary antibodies (1:500, Life Technologies). For evaluating inflammation in FIGS. 26-28 and 32-36, individual brain slices were stained for markers NeuN, IbaI, GFAP, and IL-6, in addition to DAPI and GFP, due to limited availability of spectral channels.

All images were acquired by the BZ-X810 fluorescence microscope (Keyence). Manual cell counting was performed using the Zen software (Zeiss) by a blinded observer uninformed of the experimental conditions. The DAPI positive cells in the images taken at CP were quantified by dividing the area into 16 subregions (4 by 4) using gridlines, randomly selecting 4 subregions for cell counts, and multiplying by 4 to estimate the total number of positive cells. Cells in all other images were counted individually. For the images obtained from staining for IbaI, the areas of positive expression were quantified using ImageJ by stacking the TIFF image to RGB and recording the total area above the set brightness threshold (60 for IbaI).

Methods-Estimation of the number of transduced cells-Every fifth of the 50 μm brain section (2 to 6 sections analyzed per brain) was taken to manually count the Gluc-RMA positive cells. For each section, cell density was estimated by dividing the number of counted cells over the transduction volume (the area occupying the transduced cells multiplied by the 50 μm thickness of the section). The total transduction volume in the brain was calculated using the Cavalieri method, in which the average of the transduction volumes measured in all sections was multiplied by the rostro-caudal distance between the first and the last analyzed sections. For the PV-Cre experiment, due to the limited number of visible positive cells in subsequent sections, only 1 section that showed the greatest number of cells was used for estimation (FIG. 34). Instead of using the Cavalieri method, the transduction volume was calculated by measuring the distance between the two positive cells detected at the most medial and lateral sides of the hippocampus, then using it as the diameter for calculating the sphere volume under the assumption of a spherical spread of AAVs. Finally, the number of transduced cells was calculated by multiplying the average cell density by the total transduction volume.

Methods—In vitro Fos activation-Fos activation in PC-12 was carried out by inducing with nerve growth factor (NGF). First, 18 mm-diameter glass coverslips were incubated in 10 μg ml⁻¹ of collagen IV (Corning) in PBS and coated overnight at 4° C., then dried under UV light. The coated coverslips were placed onto a 12-well plate and PC-12 was seeded at 100,000 cells per well. After 20 h, 1500 ng of plasmids encoding AAV-Fos-RMA and 3 μl of lipofectamine 2000 were used to transfect PC-12. The next day, fresh media supplemented with 100 ng ml⁻¹ of NGF was added to induce Fos activation. Then, 20 μl of media were collected at different timepoints and used for luciferase assay. After the last collection, cells on coverslips were fixed and stained against Gluc and Fos and imaged under the fluorescence microscope.

Methods—IVIS spectrum imaging—Mice were anesthetized in 1.5%-2% isoflurane in air or O₂. The body hair was removed using a hair trimmer. Mice were transferred to the IVIS spectrum imager (Perkin Elmer) while maintaining the appropriate anesthetic condition. For BLI settings, the study used open filter, large binning, F/1 aperture control, and the auto-exposure time. Researchers observed most images being generated with the 0.5 sec exposure time. Mice were injected i.v. with 12 μmol kg⁻¹ of CTZ (Nanolight Technology, #303-INJ) and immediately imaged. Images were analyzed by the Living Image software (Caliper Life Sciences) to quantify the average radiance of the upper body and the head.

Methods-Intravenous (i.v.) injection of PHP.eB-Mice were anesthetized and ophthalmic ointment (OptixCare Eye Lube Plus) was applied to both eyes to prevent corneal drying. After gently penetrating the skin along the line of the mouse's tail-vein using a catheter equipped with a 30-gauge needle until blood backflow was achieved, the catheter was secured in place using tissue glue. Through the catheter, PHP.eB AAV encoding either Gluc or Gluc-RMA under the hSyn promoter was injected at three different doses: 5.0×10⁹ vg/g, 1.2×10⁹ vg/g, and 5.0×10⁸ vg/g. Blood collection from the retro-orbital sinus was performed at 3 and 5 weeks post-delivery for luciferase assay, including for the WT mice that did not receive PHP.eB. IVIS was performed at 3^{rd} week for those mice received the highest dose (5.0×10⁹ vg/g). At 5 weeks post-delivery, mice were euthanized, perfused, and their brain, heart, kidney, and liver tissues were collected for histological imaging.

Statistical analysis—Two-tailed t test with unequal variance was used to compare two data sets. One-way ANOVA with Tukey honestly significant difference post hoc test was used to compare means between more than two data sets. Two-way ANOVA with Sidak's multiple comparison tests were used to compare data sets with two or more variables. Linear regression was used to find correlation between the plasma signal and the number of transduced neurons. All P values were determined using Prism (GraphPad Software), with the statistical significance represented as ns (not significant), *P<0.05, ** P<0.01, ***P<0.001, ****P<0.0001.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A gene expression reporter, comprising:
a released marker of activity (RMA) configured to cross a neural cell membrane and a blood brain barrier to report a gene expression in a region of a brain, the RMA comprising:
a cell secretion signaling sequence;
a detectable marker; and
a fragment crystallizable-region (Fc-region) of an antibody.

2. The gene expression reporter of claim 1, wherein the RMA is configured to undergo exocytosis to move from a respective neuron into the extracellular space.

3. The gene expression reporter of claim 1, wherein the detectable marker comprises one or more of luciferase, a fluorescent protein, or an epitope of an antibody.

4. The gene expression reporter of claim 1, wherein the Fc-region of the antibody binds to a neonatal Fc-receptor (FcRn) expressed on or in the blood brain barrier in a pH-dependent manner.

5. The gene expression reporter of claim 4, wherein Fc binds to FcRn at pH<6.5 but does not bind to FcRn at a physiological pH of 7.4.

6. The gene expression reporter of claim 1, wherein the Fc-region enables reverse transcytosis across the blood brain barrier.

7. The gene expression reporter of claim 1, wherein the RMA is detectable in the blood by one or both of a biochemical detection assay or bioluminescence imaging (BLI) techniques.

8. The gene expression reporter of claim 1, wherein the region of the brain comprises 100 neurons or less.

9. The gene expression reporter of claim 1, wherein Gaussia luciferase (Gluc) is included in the RMA and functions as both the cell secretion signaling sequence and the detectable marker.

10. The gene expression reporter of claim 9, wherein RMA comprise SEQ ID NO: 1.

11. A method for noninvasive, site-specific monitoring of expression of a gene, comprising:
causing expression of one or more synthetic released markers of activity (RMAs) at a targeted brain site of a subject, wherein each RMA comprises:
a cell secretion signaling sequence;
a detectable marker; and
a fragment crystallizable-region (Fc-region) of an antibody;
and wherein each RMA is configured to:
cross neuronal cell membranes; and
cross a blood brain barrier of the subject;
acquiring a blood sample of the subject;
performing a detection assay on the blood sample to detect and quantify presence of RMAs in the sample.

12. The method of claim 11, wherein releasing the RMAs into the blood of the subject occurs via reverse transcytosis.

13. The method of claim 11, comprising:
repeating, at one or more subsequent times, the steps of causing expression of one or more synthetic RMAs at the targeted brain site of the subject; acquiring a respective blood sample; and performing the biochemical detection assay.

14. The method of claim 13, further comprising identifying a trend or difference in expression of the gene over time.

15. The method of claim 11, wherein causing expression of one or more synthetic RMAs at a targeted brain site of a subject comprises causing expression of the one or more synthetic RMAs in one or more transduced cells and secretion of the RMAs into surrounding tissue.

16. The method of claim 11, wherein performing a detection assay on the blood sample comprises performing a biochemical detection assay.

17. The method of claim 16, wherein the biochemical detection assay comprises a multiplexed biochemical detection assay.

18. The method of claim 11, wherein performing a detection assay on the blood sample comprises performing a bioluminescence imaging (BLI) technique.

19. A signal detection system, comprising:
an assay or detection technique configured to detect a released marker of activity (RMA) present in a blood sample, the RMA comprising:
a cell secretion signaling sequence;
a detectable marker; and
a fragment crystallizable-region (Fc-region) of an antibody;
wherein the presence or quantity of RMA in the blood sample corresponds to expression of a gene in a targeted region of a brain.

20. The signal detection system of claim 19, wherein the assay or detection technique comprises a biochemical detection assay or a bioluminescence imaging (BLI) technique.
